(19) United States
(12) Patent Application Publication
(10) Pub. No.: US 2011/0091884 A1
(43) Pub. Date: Apr. 21, 2011

(54) Method for Analysis of DNA Methylation

(76) Inventor: Naomi Yamakawa, Kawagoe-shi (JP)

(21) Appl. No.: 12/910,870

(22) Filed: Oct. 25, 2010

Related U.S. Application Data
(63) Continuation of application No. PCT/JP2009/058199, filed on Apr. 24, 2009.

(30) Foreign Application Priority Data
Apr. 25, 2008 (JP) 2008-115355

(51) Int. Cl. C12Q 1/68; C12P 19/34

(52) U.S. Cl. 435/6; 435/91.52

(57) ABSTRACT

Provided is a method for analysis of DNA methylation. A first method comprises: (1) a step in which analyte DNA is digested with methylation-insensitive restriction enzyme(s) that produce overhanging end(s) and that contain methylated cytosine or methylatable cytosine in recognition sequence(s) thereof; (2) a step in which adaptor(s) capable of regenerating recognition sequence(s) of the aforesaid methylation-insensitive restriction enzyme(s) is/are ligated to both ends of DNA fragment(s) obtained at the aforesaid step (1); and (3) a step in which DNA construct(s) obtained at the aforesaid step (2) is/are digested with methylation-sensitive restriction enzyme(s) that recognize the same recognition sequence(s) as the aforesaid methylation-insensitive restriction enzyme(s). A second method comprises: (1) a step in which analyte DNA is digested with restriction enzyme(s); (2) a step in which adaptor(s) is/are ligated to both ends of DNA fragment(s) obtained at the aforesaid step (1); and (3) a step in which DNA construct(s) obtained at the aforesaid step (2) is/are digested with methylation-dependent restriction enzyme(s).

### CROSS-REFERENCE TO RELATED APPLICATIONS AND INCORPORATION BY REFERENCE

This application is a continuation of, and claims benefit of priority under 35 USC 120 to, copending International Application No PCT/JP2009/058199, entitled “Method for Analysis of DNA Methylation”, filed 24 Apr. 2009, the content of which is incorporated herein in its entirety by reference; and further, claims benefit of priority under 35 USC 119 to Japanese Patent Application No 2008-115355, entitled “Method for Analysis of DNA Methylation”, filed 25 Apr. 2008, the content of which is incorporated herein in its entirety by reference. All patent and nonpatent literature, and all protocols and procedures, referred to in the present specification, including those referred to in the working examples, are incorporated herein in their entirety by reference.

### TECHNICAL FIELD

The present invention relates to a method for analysis of DNA methylation.

### BACKGROUND ART

A portion of the cytosine in the genomic DNA of higher organisms is methylated, this DNA modification together with histone modification playing an extremely important role in ontogeny. Such epigenetic regulation may be subject to reprogramming as a result of fertilization or the like, such that the cell reacquires capacity for ontogenetic expression, i.e., pluripotency. A method for artificially inducing such reprogramming has recently been discovered by Takahashi et al, a cell produced in this way being referred as an iPS cell (induced pluripotent cell; Nonpatent Literature Reference No. 1). While the level of DNA methylation in mammalian sperm and ova is roughly close to that in biological tissue, sperm DNA is actively demethylated within an extremely short time immediately following fertilization. On the other hand, ovum DNA, after being united with sperm DNA, gradually undergoes demethylation in accompaniment to cell division until preimplantation, the level of methylation being at its lowest immediately prior to implantation. In this way, DNA initialization is completed. Embryonic stem cells (ES cells), widely utilized at the present time to produce artificially mutated animals, are cells that have been isolated from fertilized eggs in which this initialization is completed, these cells themselves possessing the pluripotency that permits differentiation to the point of individual formation.

ES cells having such pluripotency are capable of being induced to differentiate in various ways when cultured. Accordingly, if target cells can be produced from human ES cells, it should be possible to transplant these into the human body to effect fundamental treatment of disease. However, because histocompatibility varies among individuals, when cells produced by induced differentiation from a particular ES cell are transplanted into a patient, it is essential following transplantation of cells that ongoing immunosuppression be carried out through administration of drugs. In contrast, iPS cells permit immunological problems to be avoided inasmuch as they permit production of pluripotent stem cells from the patient's own tissue, and varied attempts are currently underway to realize the potential thereof in cell transplantation medicine (Nonpatent Literature Reference No. 2, Nonpatent Literature Reference No. 3).

When normal cells are cultured over multiple generations, the cells ordinarily undergo aging in the same fashion as the individual. In terms of specific phenomena, shortening of telomeres and aberrations in DNA methylation are observed. For example, where aberration in DNA methylation occurs at a cancer-critical gene, this might cause the cell to undergo tumorigenesis. Of course, it is also established that such cell alterations may also be caused by base sequence mutations and chromosomal translocations. To realize the potential of cell transplantation medicine, such undesirable changes in genomic DNA that occur in culture must be prevented, or cells in which such changes have occurred must be eliminated from the cells that will be used for transplantation. For this reason, it is necessary to analyze DNA methylation over the entire genome with respect to all aspects important to cell production so as to ensure safety. For example, iPS cells are produced by introducing three or four types of gene into cells separated from normal human tissue, and it has been difficult in the conventional art to determine whether the separated iPS cells have acquired full pluripotency. For example, confirmation of totipotency in a mouse iPS cell may be carried out by injecting the iPS cell so produced into a fertilized mouse egg, and checking to see whether the iPS cell causes chimerism to be exhibited throughout all tissue of the mouse individual that results. Furthermore, while it is not a perfect guarantee of pluripotency, evaluation is sometimes carried out by administering the iPS cell to a nude mouse and evaluating pluripotency based on teratocarcinoma formation. However, analysis of formation of chimera with animals in the same fashion as done for mice would be an impracticable analytical technique for confirmation of pluripotency of human iPS cells due to the ethical problems associated therewith. Under such circumstances, genome-wide analysis of DNA methylation is an extremely important analytical technique, it being no exaggeration to say that there is at present no alternative technique available. During the process of differentiation of pluripotent stem cells, the pattern of DNA methylation is accurately controlled to define various cells types. Conversely, analysis of this methylation pattern therefore makes it possible to identify cell type and also to analyze whether a cell possesses full pluripotency. That is, cell phenotype can be known in detail from the DNA methylation pattern.

When cells are cultured over successive generations, mutations are found to occur with certain frequencies. For example, when normal mouse cells are grown over successive generations, immortalized cells tend to appear with a certain constant frequency. This might be said to be a type of malignant transformation. Chromosomal aberrations, base-level mutations at cancer-critical genes, or aberrations in DNA methylation are thought to contribute to appearance of such cells. Among these, DNA methylation can be said to be a sensitive analytic technique for early diagnosis of malignant transformation in cells, because aberrations are observed with high frequency at core promoter regions of cancer-critical genes and CpG islands during the initial stages of malignant transformation of cells.

As previously mentioned, genome-wide analysis of DNA methylation is an extremely useful analytical technique for learning about cell state, and possesses a domain that is irreplaceable by any other analytic method. It is therefore an essential analytical technique in the field of cell transplantation medicine. Accordingly, toward clinical application of this analytical technique, there is eager interest in an analytical technique that would employ a simple analytic procedure and that would permit detailed analysis over the entire genome.

Among DNA methylation analysis methods employing DNA arrays, a number of methods employing anti-5-methylcytosine antibody, such as the MeDIP technique (Nonpatent Literature Reference No. 4), the MONIC technique (Patent Literature Reference No. 1), and so forth, have been reported in the known art. In contradistinction to methods employing antibodies, methods employing restriction enzyme properties have also been reported; e.g., the DMH technique (Nonpatent Literature Reference No. 5) and the MIAMI method (Nonpatent Literature Reference No. 6).

### PRIOR ART REFERENCES

### Patent Literature

Patent Literature Reference No. 1: International Patent Application Publication No. 2005/080565

### Nonpatent Literature

Nonpatent Literature Reference No. 1: Cell, (U.S.), 2006, Vol. 126, pp. 663-676
Nonpatent Literature Reference No. 2: Cell, (U.S.), 2007, Vol. 131, pp. 861-872
Nonpatent Literature Reference No. 3: Nature Biotechnology, (U.S.), 2008, Vol. 26, pp. 101-106
Nonpatent Literature Reference No. 4: Nature Genetics, (U.S.), 2005, Vol. 37, pp. 853-862
Nonpatent Literature Reference No. 5: Cancer Research, (U.S.), 2001, Vol. 61, pp. 8375-8380
Nonpatent Literature Reference No. 6: Oncogene, (U.K.), 2006, Vol. 25, pp. 3059-3064

### SUMMARY OF INVENTION

### Problems to be Solved by Invention

However, the aforementioned known methods employing anti-5-methylcytosine antibody have been deficient from the standpoints that several days are required for complete analysis; antibodies and/or other special reagents are required; and, due to the fact that these methods employ immunoprecipitation through use of antibodies, target DNA recovery rates are low, making large quantities of DNA samples necessary for analysis. When induced differentiation is carried out on stem cells in culture and some of those cells are used for analysis, because the number of cells that can be used for analysis is limited, it is necessary to use an analytical method having high sensitivity. Accordingly, a method that would permit analysis of DNA even in quantities on the order of a nanogram is desired.

On the other hand, with the aforementioned known methods employing restriction enzyme properties, because target DNA recovery rates are high, it is possible to carry out analysis based on small samples. However, with such conventional methods, because methylation-sensitive restriction enzymes are used at an early stage, cleavage sites within the genome are methylation-dependent, and there are few genomic cleavage sites. This therefore limits the analytical resolution of conventional analytical methods. Methods employing DNA arrays also include methods in which DNA is chemically treated by hydrogen sulfite ion (bisulfite) and array analysis is thereafter carried out, but because these are such that unmethylated cytosine in DNA is converted to uracil, and this is converted to thymine during the PCR amplification reaction, there has been the disadvantage that complexity of DNA base sequences is reduced, as a result of which homology of any given genomic region with respect to other regions is increased, increasing the likelihood that it will be impossible, using the short DNA oligos employed in DNA arrays, to detect a particular gene fragment with specificity.

### Means for Solving the Problems

In light of problems such as the aforementioned in the conventional art, and as a result of diligent efforts by the present inventor to develop high-resolution and high-sensitivity methods for analysis of genomic DNA methylation, the techniques of the present invention were developed. Characteristic of the present invention is ability to analyze not only methylation at both overhanging ends of DNA fragments produced by fragmentation into small pieces (meaning high-resolution) by methylation-insensitive restriction enzymes but also methylation at interior regions of DNA fragments. In accordance with conventional methods, a methylation-sensitive restriction enzyme is first used to fragment DNA, adaptors are ligated to both ends, this is then further digested with a methylation-insensitive restriction enzyme, and methylated sites are identified. Accordingly, there are many gene promoter regions that will be unanalyzable using conventional methods. Accordingly, it was not possible to analyze all genes. In accordance with the present invention, to achieve high-resolution analysis, a methylation-insensitive restriction enzyme is first used to fragment genomic DNA into small pieces, with various strategies being employed to permit analysis of methylation at both ends of these DNA fragments. As a result, it is possible to dramatically improve analytic resolution as compared with conventional methods, making it possible to carry out analysis with respect to nearly all gene core promoter regions and CpG islands. Furthermore, in accordance with the present invention, following adaptor ligation, redigestion with methylation-sensitive restriction enzyme is carried out, while at the same time, an identical sample is digested in similar fashion with methylation-insensitive restriction enzyme, these both being subjected to LM-PCR (ligation-mediated PCR) and thereafter compared by means of electrophoresis so that complete removal of adaptors can be confirmed. That is, this makes it possible to evaluate whether or not analytic results were accurately determined based on restriction enzyme methylation sensitivity alone. Such a technique is not present within known conventional methods. When analyzing and evaluating clinical samples, because it is extremely important that the analytic systems employed be such as will permit quality checks to be carried out at each of the various analytic operations, the benefit afforded by the present invention addresses such social demands.

Restriction enzyme digest and adaptor ligation do not ordinarily proceed with 100% efficiency, and DNA ends that are undigested and/or unligated by adaptors can become mixed with analytic system contents. While admixture by such incomplete treatment products would not present a problem for ordinary molecular biological experiments, in situations where quantitative analysis of methylation of DNA is performed, such as is the case with methylation analysis, it may alter those analytic results. That is, it may cause methylated sites to be evaluated as unmethylated sites, and conversely, may cause unmethylated sites to be evaluated as methylated sites. In accordance with present invention, such problems are also addressed, and measures have been developed for solution thereof.

Furthermore, while the present invention may make use of the known art of LM-PCR, optimal conditions for specific amplification of adaptor-ligated DNA have been discovered. In most of the known art, it is usually the case that oligo DNA at one member of a double-stranded adaptor is used as primer to carry out LM-PCR. However, studies made by the present inventor have confirmed that in almost all cases DNA that is unligated by adaptor(s) is also amplified together therewith in nonspecific fashion. It is thought that this could be due to the fact that in LM-PCR the specificity exhibited during PCR must be ensured by only a single oligo DNA primer comprising from 10 or so to 20 or so bases. That is, in light of the wide variety of base sequences in the genome, there will be many locations having homology with respect to a primer that is as short as the aforementioned, and so even without ligation of an adaptor, it is speculated that the primer hybridizes to regions having high homology with the primer, and that this is why nonspecific amplicons are observed.

A further characteristic of the present invention is that it permits occurrence of such nonspecific amplification to be confirmed in mid-analysis, and the fact that such a quality check has been made possible is what first made possible embodiment(s) of the present invention. Because the aforementioned known art does not include such an operation for verification of LM-PCR specificity, it does not afford an opportunity to judge the reliability of analytic results until the final results of analysis are seen. That is, it does not permit establishment of steps for monitoring occurrence of nonspecific amplification during LM-PCR or other such amplification steps. This is because, since a methylation-sensitive restriction enzyme is used at the beginning, DNA fragments obtained in this fashion through use of a methylation-sensitive restriction enzyme have a high probability of containing sites at the interior thereof that would be cleaved by a methylation-insensitive restriction enzyme, and so it would be fundamentally impossible to carry out verification of amplification specificity during LM-PCR following digest of adaptor(s) with methylation-insensitive restriction enzyme(s) as in the instant invention.

Such problems were first made solvable by techniques in accordance with the present invention, being issues not amenable to solution through combination of the simplicity of the known art.

The present invention relates to

[1] a method for analyzing methylation of DNA analyte, the method characterized in that it comprises:
(1) a step in which analyte DNA is digested with a methylation-insensitive restriction enzyme that produces an overhanging end and that contains methylated cytosine or methylatable cytosine in a recognition sequence thereof;
(2) a step in which an adaptor capable of regenerating a recognition sequence of the aforesaid methylation-insensitive restriction enzyme is ligated to both ends of a DNA fragment obtained at the aforesaid step (1); and
(3) a step in which a DNA construct obtained at the aforesaid step (2) is digested with a methylation-sensitive restriction enzyme that recognizes the same recognition sequence as the aforesaid methylation-insensitive restriction enzyme;

[2] a method according to [1] further comprising:
a step in which a digest product obtained at step (3) at [1], or a DNA amplicon obtained by amplification using an amplification primer capable of hybridization with the adaptor referred to at step (2) at [1] with the aforesaid digest product serving as template, is analyzed;

[3] a method according to [2] further comprising:
a step in which a DNA construct obtained at step (2) at [1] is digested with a methylation-insensitive restriction enzyme that recognizes the same recognition sequence as the methylation-insensitive restriction enzyme referred to at step (1) at [1]; and
a step in which the digest product obtained at the aforesaid step, or a DNA amplicon obtained by amplification using the amplification primer referred to at [2] with the aforesaid digest product serving as template, is analyzed;

[4] a method according to [2] or [3] further comprising:
a step in which the DNA construct obtained at step (2) at [1], or a DNA amplicon obtained by amplification using the amplification primer referred to at [2] with the aforesaid DNA construct serving as template, is analyzed;

[5] a method according to [1] wherein the adaptor referred to at step (2) at [1] is labeled with a substance capable of selective binding;

[6] a method according to [5] further comprising:
(4a) a step in which a digest product obtained at step (3) at [1] is separated based on presence or absence of the aforesaid label; and
(5a) a step in which one or both separated DNA fragments is or are analyzed;

[7] a method according to [5] wherein the enzyme digest treatment referred to at step (3) at [1] is carried out while the DNA construct is captured by a carrier by way of the aforesaid labeled substance, and further comprising:
(4b) a step in which the immobilizing carrier used to carry out the enzyme digest treatment of step (3) is washed, and separation into a DNA fragment captured by the carrier and a DNA fragment liberated from the carrier is carried out; and
(5b) a step in which one or both separated DNA fragments is or are analyzed;

[8] a method according to [1] through [7] further comprising:
a step in which a DNA construct obtained at step (2) at [1] is digested with a methylation-dependent restriction enzyme; and
a step in which the digest product obtained at the aforesaid step, or a DNA amplicon obtained by amplification using the amplification primer referred to at [2] with the aforesaid digest product serving as template, is analyzed;

[9] a method for using the method at [8] to determine whether respective DNA fragments obtained at step (1) at [1] are:
(a) DNA exhibiting sensitivity with respect to a methylation-dependent restriction enzyme, and having methylated cytosine present in a restriction enzyme recognition sequence at each of the two ends thereof;
(b) DNA exhibiting digest resistance with respect to a methylation-dependent restriction enzyme, and having methylated cytosine present in a restriction enzyme recognition sequence at each of the two ends thereof;
(c) DNA exhibiting sensitivity with respect to a methylation-dependent restriction enzyme, and having methylated cytosine present in a restriction enzyme recognition sequence at only one of the two ends thereof or having methylated cytosine present at neither of the two ends thereof;
(d) DNA exhibiting digest resistance with respect to a methylation-dependent restriction enzyme, and having methylated cytosine present in a restriction enzyme recognition sequence at only one of the two ends thereof or having methylated cytosine present at neither of the two ends thereof;

[10] a method according to [1] through [9] wherein DNA that has been treated in advance with single-strand specific nuclease is used as the analyte DNA employed at step (1);

[11] a method of manufacturing:
(I′) a DNA group obtained by ligation of one or more DNA fragments, each of which DNA fragment has at each of the two ends thereof a recognition sequence containing methylated cytosine or methylatable cytosine, methylated cytosine being present at each of the two recognition sequences;
(II′) a DNA group obtained by ligation of one or more DNA fragments, each of which DNA fragment has at each of the two ends thereof a recognition sequence containing methylated cytosine or methylatable cytosine, methylated cytosine being present at only one of the two recognition sequences; and/or
(III′) a DNA group obtained by ligation of one or more DNA fragments, each of which DNA fragment has at each of the two ends thereof a recognition sequence containing methylated cytosine or methylatable cytosine, methylated cytosine being present at neither of the two recognition sequences;
the method characterized in that it comprises:
(A) a step in which a method according to [1] through [10] is used to acquire:
(i) a DNA fragment group containing only DNA fragments, each of which DNA fragment has at each of the two ends thereof a recognition sequence containing methylated cytosine or methylatable cytosine, methylated cytosine being present at each of the two recognition sequences;
(ii) a DNA fragment group containing only DNA fragments, each of which DNA fragment has at each of the two ends thereof a recognition sequence containing methylated cytosine or methylatable cytosine, methylated cytosine being present at only one of the two recognition sequences; and/or
(iii) a DNA fragment group containing only DNA fragments, each of which DNA fragment has at each of the two ends thereof a recognition sequence containing methylated cytosine or methylatable cytosine, methylated cytosine being present at neither of the two recognition sequences; and
(B) a step in which the acquired DNA fragment group or groups is or are digested with the methylation-insensitive restriction enzyme referred to at [1] to cleave and eliminate all terminal adaptors, and ligation is thereafter carried out;

[12] a method of manufacturing:
(I) a circular DNA group containing only circular DNA obtained by ligation of one or more DNA fragments, each of which DNA fragment has at each of the two ends thereof a recognition sequence containing methylated cytosine or methylatable cytosine, methylated cytosine being present at each of the two recognition sequences;
(II) a circular DNA group containing only circular DNA obtained by ligation of one or more DNA fragments, each of which DNA fragment has at each of the two ends thereof a recognition sequence containing methylated cytosine or methylatable cytosine, methylated cytosine being present at only one of the two recognition sequences; and/or
(III) a circular DNA group containing only circular DNA obtained by ligation of one or more DNA fragments, each of which DNA fragment has at each of the two ends thereof a recognition sequence containing methylated cytosine or methylatable cytosine, methylated cytosine being present at neither of the two recognition sequences;
wherein exonuclease treatment is further carried out following the aforesaid ligation;

[13] a method of manufacturing:
(I′) a DNA group obtained by ligation of one or more DNA fragments, each of which DNA fragment has at each of the two ends thereof a recognition sequence containing methylated cytosine or methylatable cytosine, methylated cytosine being present at each of the two recognition sequences;
(IIa′) a DNA group obtained by ligation of one or more DNA fragments, each of which DNA fragment has at each of the two ends thereof a recognition sequence containing methylated cytosine or methylatable cytosine, methylated cytosine being present at only the upstream recognition sequence;
(IIb′) a DNA group obtained by ligation of one or more DNA fragments, each of which DNA fragment has at each of the two ends thereof a recognition sequence containing methylated cytosine or methylatable cytosine, methylated cytosine being present at only the downstream recognition sequence; and/or
(III′) a DNA group obtained by ligation of one or more DNA fragments, each of which DNA fragment has at each of the two ends thereof a recognition sequence containing methylated cytosine or methylatable cytosine, methylated cytosine being present at neither of the two recognition sequences;
the method characterized in that it comprises:
(A) a step in which a method according to [1] through [10] is used to acquire:
(i) a DNA fragment group containing only DNA fragments, each of which DNA fragment has at each of the two ends thereof a recognition sequence containing methylated cytosine or methylatable cytosine, methylated cytosine being present at each of the two recognition sequences;
(iia) a DNA fragment group containing only DNA fragments, each of which DNA fragment has at each of the two ends thereof a recognition sequence containing methylated cytosine or methylatable cytosine, methylated cytosine being present at only the upstream recognition sequence;
(iib) a DNA fragment group containing only DNA fragments, each of which DNA fragment has at each of the two ends thereof a recognition sequence containing methylated cytosine or methylatable cytosine, methylated cytosine being present at only the downstream recognition sequence; and/or
(iii) a DNA fragment group containing only DNA fragments, each of which DNA fragment has at each of the two ends thereof a recognition sequence containing methylated cytosine or methylatable cytosine, methylated cytosine being present at neither of the two recognition sequences; and
(B) a step in which the acquired DNA fragment group or groups is or are digested with the methylation-insensitive restriction enzyme referred to at [1] to cleave and eliminate all terminal adaptors, and ligation is thereafter carried out;

[14] a method of manufacturing:
(I) a circular DNA group containing only circular DNA obtained by ligation of one or more DNA fragments, each of which DNA fragment has at each of the two ends thereof a recognition sequence containing methylated cytosine or methylatable cytosine, methylated cytosine being present at each of the two recognition sequences;
(IIa) a circular DNA group containing only circular DNA obtained by ligation of one or more DNA fragments, each of which DNA fragment has at each of the two ends thereof a recognition sequence containing methylated cytosine or methylatable cytosine, methylated cytosine being present at only the upstream recognition sequence;
(IIb) a circular DNA group containing only circular DNA obtained by ligation of one or more DNA fragments, each of which DNA fragment has at each of the two ends thereof a recognition sequence containing methylated cytosine or methylatable cytosine, methylated cytosine being present at only the downstream recognition sequence; and/or
(III) a circular DNA group containing only circular DNA obtained by ligation of one or more DNA fragments, each of which DNA fragment has at each of the two ends thereof a recognition sequence containing methylated cytosine or methylatable cytosine, methylated cytosine being present at neither of the two recognition sequences;
wherein exonuclease treatment is further carried out following the aforesaid ligation;

[15] a DNA group characterized in that it contains only DNA which is one of:
(1) DNA obtained by ligation of one or more DNA fragments, each of which DNA fragment has at each of the two ends thereof a recognition sequence containing methylated cytosine or methylatable cytosine, methylated cytosine being present at each of the two recognition sequences;
(2) DNA obtained by ligation of one or more DNA fragments, each of which DNA fragment has at each of the two ends thereof a recognition sequence containing methylated cytosine or methylatable cytosine, methylated cytosine being present at only one of the two recognition sequences; or
(3) DNA obtained by ligation of one or more DNA fragments, each of which DNA fragment has at each of the two ends thereof a recognition sequence containing methylated cytosine or methylatable cytosine, methylated cytosine being present at neither of the two recognition sequences;

[16] a DNA group characterized in that it contains only DNA which is one of:
(1) DNA obtained by ligation of one or more DNA fragments, each of which DNA fragment has at each of the two ends thereof a recognition sequence containing methylated cytosine or methylatable cytosine, methylated cytosine being present at each of the two recognition sequences;
(2a) DNA obtained by ligation of one or more DNA fragments, each of which DNA fragment has at each of the two ends thereof a recognition sequence containing methylated cytosine or methylatable cytosine, methylated cytosine being present at only the upstream recognition sequence;
(2b) DNA obtained by ligation of one or more DNA fragments, each of which DNA fragment has at each of the two ends thereof a recognition sequence containing methylated cytosine or methylatable cytosine, methylated cytosine being present at only the downstream recognition sequence; or
(3) DNA obtained by ligation of one or more DNA fragments, each of which DNA fragment has at each of the two ends thereof a recognition sequence containing methylated cytosine or methylatable cytosine, methylated cytosine being present at neither of the two recognition sequences;

[17] a circular DNA group characterized in that it contains only circular DNA which is one of:
(1) circular DNA obtained by ligation of one or more DNA fragments, each of which DNA fragment has at each of the two ends thereof a recognition sequence containing methylated cytosine or methylatable cytosine, methylated cytosine being present at each of the two recognition sequences;
(2) circular DNA obtained by ligation of one or more DNA fragments, each of which DNA fragment has at each of the two ends thereof a recognition sequence containing methylated cytosine or methylatable cytosine, methylated cytosine being present at only one of the two recognition sequences; or
(3) circular DNA obtained by ligation of one or more DNA fragments, each of which DNA fragment has at each of the two ends thereof a recognition sequence containing methylated cytosine or methylatable cytosine, methylated cytosine being present at neither of the two recognition sequences;

[18] a circular DNA group characterized in that it contains only circular DNA which is one of:
(1) circular DNA obtained by ligation of one or more DNA fragments, each of which DNA fragment has at each of the two ends thereof a recognition sequence containing methylated cytosine or methylatable cytosine, methylated cytosine being present at each of the two recognition sequences;
(2a) circular DNA obtained by ligation of one or more DNA fragments, each of which DNA fragment has at each of the two ends thereof a recognition sequence containing methylated cytosine or methylatable cytosine, methylated cytosine being present at only the upstream recognition sequence;
(2b) circular DNA obtained by ligation of one or more DNA fragments, each of which DNA fragment has at each of the two ends thereof a recognition sequence containing methylated cytosine or methylatable cytosine, methylated cytosine being present at only the downstream recognition sequence; or
(3) circular DNA obtained by ligation of one or more DNA fragments, each of which DNA fragment has at each of the two ends thereof a recognition sequence containing methylated cytosine or methylatable cytosine, methylated cytosine being present at neither of the two recognition sequences;

[19] a linear DNA group characterized in that it contains only DNA fragments which are either:
DNA fragments, each of which DNA fragment has at each of the two ends thereof a recognition sequence containing methylated cytosine or methylatable cytosine, methylated cytosine being present at only the upstream recognition sequence; or
DNA fragments, each of which DNA fragment has at each of the two ends thereof a recognition sequence containing methylated cytosine or methylatable cytosine, methylated cytosine being present at only the downstream recognition sequence;

[20] a linear DNA group according to [19] wherein the same or different adaptors are ligated to the two ends of each of the aforesaid DNA fragments;

[21] a DNA array characterized in that nucleic acids respectively capable of hybridization with all or any portion of the DNA fragments contained in a DNA fragment group according to [19] or [20] are arranged on a carrier;

[22] a method for analyzing methylation of DNA analyte, the method characterized in that a DNA amplicon obtained by amplification by means of strand displacement DNA polymerase using a random primer with a circular DNA group according to [17] or [18] serving as template is analyzed;

[23] a DNA array characterized in that nucleic acids respectively capable of hybridization with all or any portion of the DNA at (a) through (d) at [9] are arranged on a carrier;

[24] a DNA group made up of only DNA fragments that exhibit resistance with respect to a methylation-dependent restriction enzyme and that have restriction enzyme recognition sequences at both ends, wherein the aforesaid recognition sequences are all the same sequence;

[25] a method for analyzing methylation of DNA analyte, the method characterized in that it comprises:
(1) a step in which analyte DNA is digested with a restriction enzyme;
(2) a step in which an adaptor is ligated to both ends of a DNA fragment obtained at the aforesaid step (1); and
(3) a step in which a DNA construct obtained at the aforesaid step (2) is digested with a methylation-dependent restriction enzyme;

[26] a method according to [25] further comprising:
a step in which a digest product obtained at step (3) at [25], or a DNA amplicon obtained by amplification using an amplification primer capable of hybridization with the adaptor referred to at step (2) at [25] with the aforesaid digest product serving as template, is analyzed;

[27] a method according to [26] wherein the aforesaid digest product serving as template is pretreated with a double-stranded DNA-digesting 5′ to 3′ exonuclease, or a double-stranded DNA-digesting 5′ to 3′ exonuclease and a single-stranded-DNA-specific exonuclease;

[28] a method for using the method at [26] or [27] to determine whether respective DNA fragments obtained at step (1) at [25] are:
(a) DNA exhibiting digest resistance with respect to a methylation-dependent restriction enzyme;
(b) DNA exhibiting sensitivity with respect to a methylation-dependent restriction enzyme;

[29] a method for using the method at [26] or [27] to determine at least interior region methylation level of respective DNA fragments obtained at step (1) at [25];

[30] a method according to [29] wherein an adaptor that does not form methylation-dependent restriction enzyme recognition sites at DNA fragment end regions is used as the adaptor referred to at [25] to determine only interior region methylation level;

[31] a method according to [29] wherein an adaptor that forms methylation-dependent restriction enzyme recognition sites at DNA fragment end regions is used as the adaptor referred to at [25] to determine overall methylation level at interior and end regions;

[32] a DNA array characterized in that nucleic acids respectively capable of hybridization with all or any portion of the DNA at (a) and (b) at [28] are arranged on a carrier;

[33] a method of manufacturing a DNA group, the method characterized in that it comprises:
(1) a step in which analyte DNA is digested with a restriction enzyme;
(2) a step in which an adaptor capable of regenerating a recognition sequence of the aforesaid restriction enzyme is ligated to both ends of a DNA fragment obtained at the aforesaid step (1);
(3) a step in which a DNA construct obtained at the aforesaid step (2) is digested with a methylation-dependent restriction enzyme; and
(4) a step in which an amplification primer capable of hybridization with the adaptor referred to in the aforesaid step (2) is used to amplify DNA, with a digest product obtained at the aforesaid step (3) serving as template;
wherein the DNA group is made up of only DNA fragments that exhibit resistance with respect to a methylation-dependent restriction enzyme and that have at each of the two ends thereof a recognition sequence of the restriction enzyme referred to at the aforesaid step (1);

[34] a DNA group made up of only DNA fragments that exhibit resistance with respect to a methylation-dependent restriction enzyme and that have restriction enzyme recognition sequences at both ends, wherein the aforesaid recognition sequences are all the same sequence;

[35] a DNA array characterized in that nucleic acids respectively capable of hybridization with all or any portion of the DNA fragments contained in the DNA fragment group according to [34] are arranged on a carrier;

[36] a method according to [25] wherein
the aforesaid adaptor is made up of combination of an oligonucleotide imparted with exonuclease resistance, and an oligonucleotide having a quencher and a fluorescent label; and
a digest product obtained at step (3) at [25] is treated with a double-stranded DNA-digesting exonuclease, methylation level being determined by measuring fluorescence thereof;

[37] a method for analyzing methylation of DNA analyte, the method characterized in that it comprises:
(1) a step in which analyte DNA is digested with a restriction enzyme;
(2) a step in which a DNA fragment obtained at the aforesaid step (1) is circularized; and
(3) a step in which circular DNA obtained at the aforesaid step (1) is digested with an MD restriction enzyme;

[38] a method according to [37] further comprising:
a step in which a digest product obtained at step (3) at [37], or a DNA amplicon obtained by amplifying only circular DNA with the aforesaid digest product serving as template, is analyzed;

[39] a circular DNA group characterized in that it is made up of only circular DNA not having methylation-dependent restriction enzyme recognition sites;

[40] a circular DNA group according to [39] that is made up of only circular DNA completely without any PumC sequences;

[41] a DNA array characterized in that nucleic acids respectively capable of hybridization with all or any portion of the DNA fragments contained in a circular DNA group according to [39] or [40] are arranged on a carrier;

[42] a method of manufacturing a circular DNA group according to [39] or [40], the method characterized in that it comprises:
(1) a step in which analyte DNA is digested with a restriction enzyme;
(2) a step in which a DNA fragment obtained at the aforesaid step (1) is circularized;
(3) a step in which circular DNA obtained at the aforesaid step (1) is digested with an MD restriction enzyme; and
(4) a step in which only circular DNA is amplified, with a digest product obtained at the aforesaid step (3) serving as template;

[43] a method according to any one of [25] through [31], [33], [36] through [38], and [42] wherein DNA that has been treated in advance with single-strand specific nuclease is used as the analyte DNA employed at step (1).

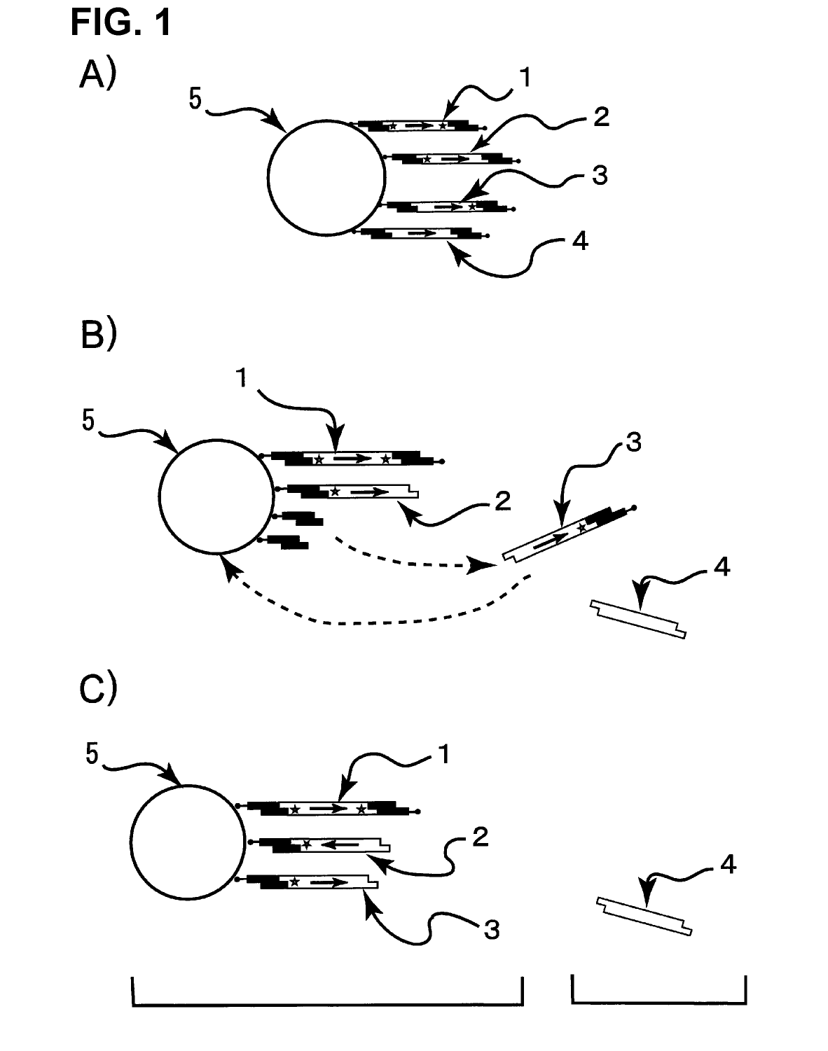

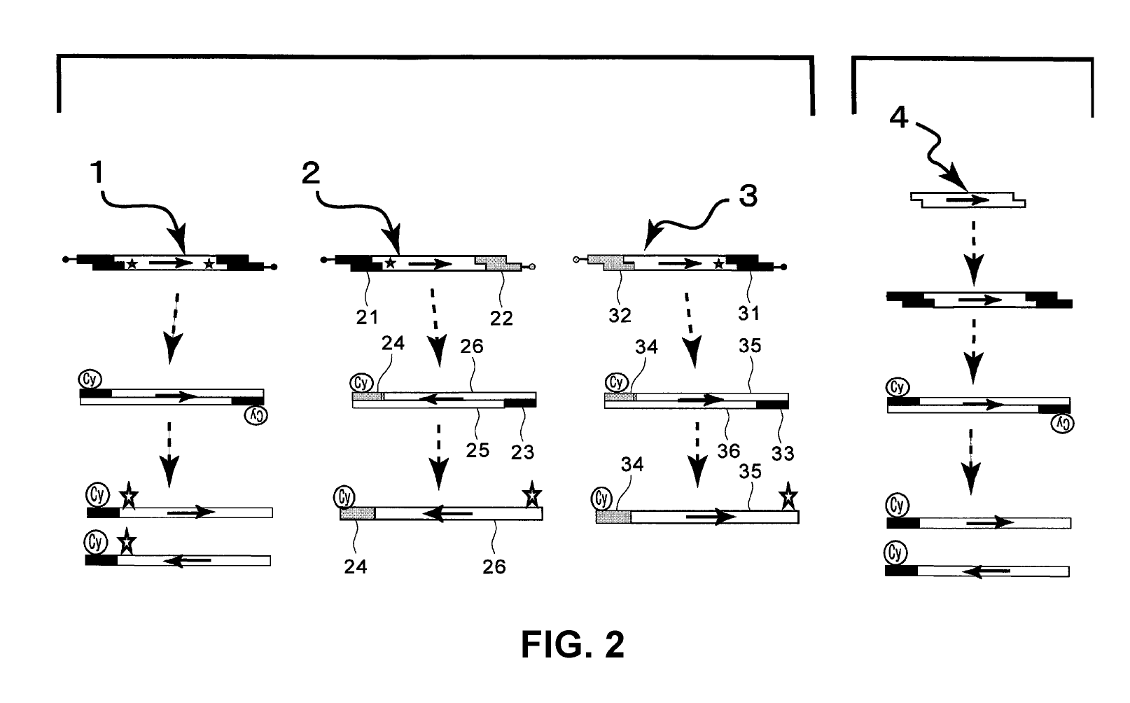

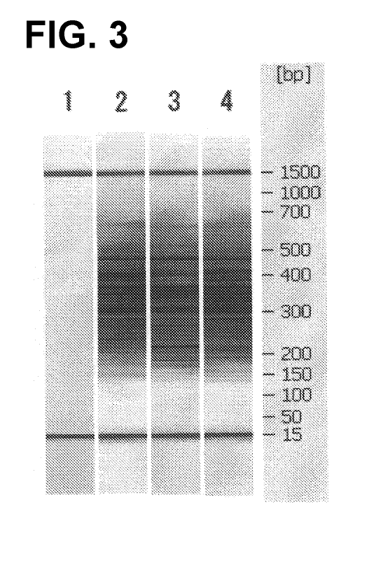

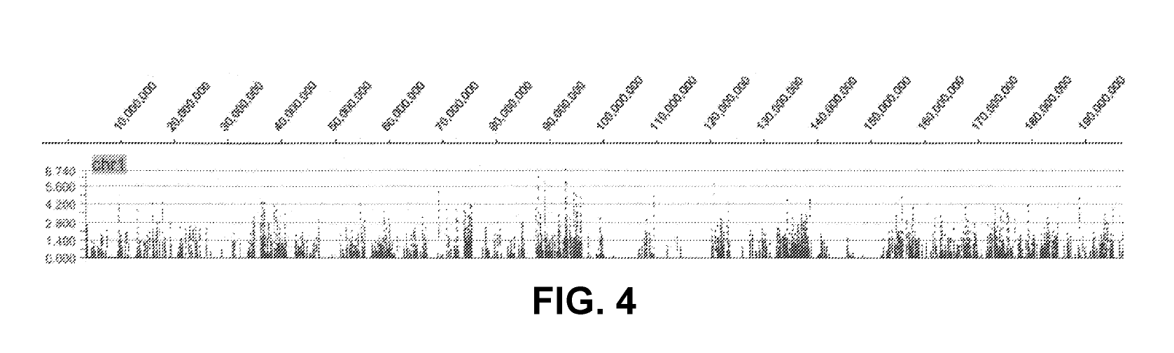

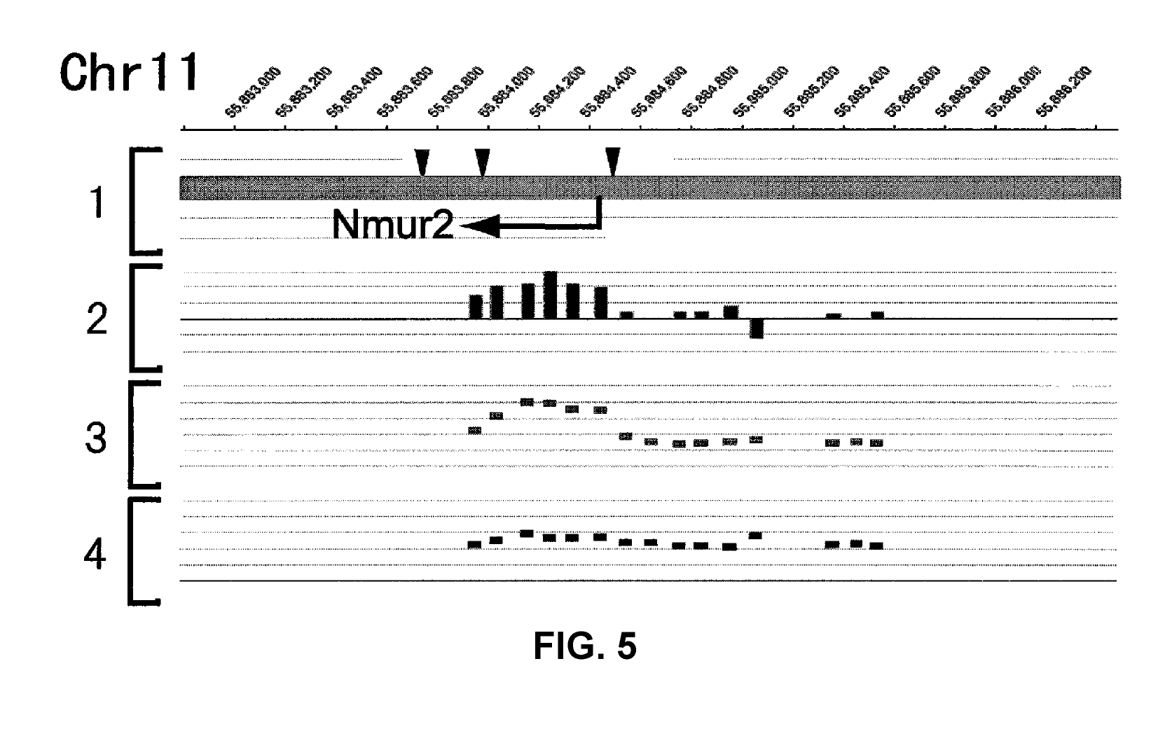

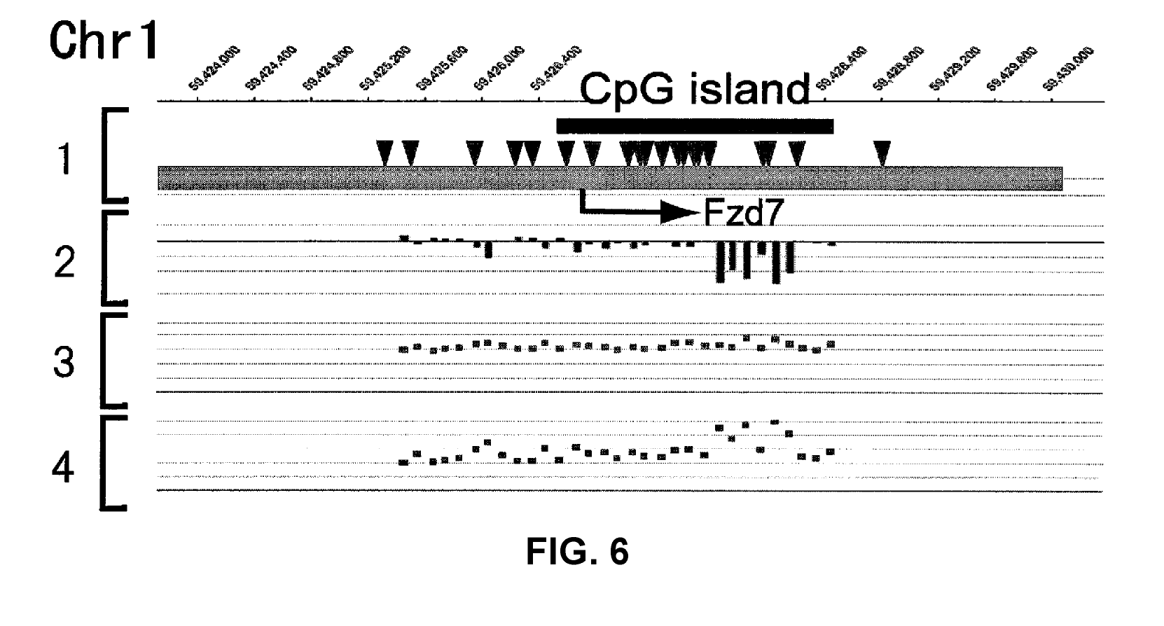

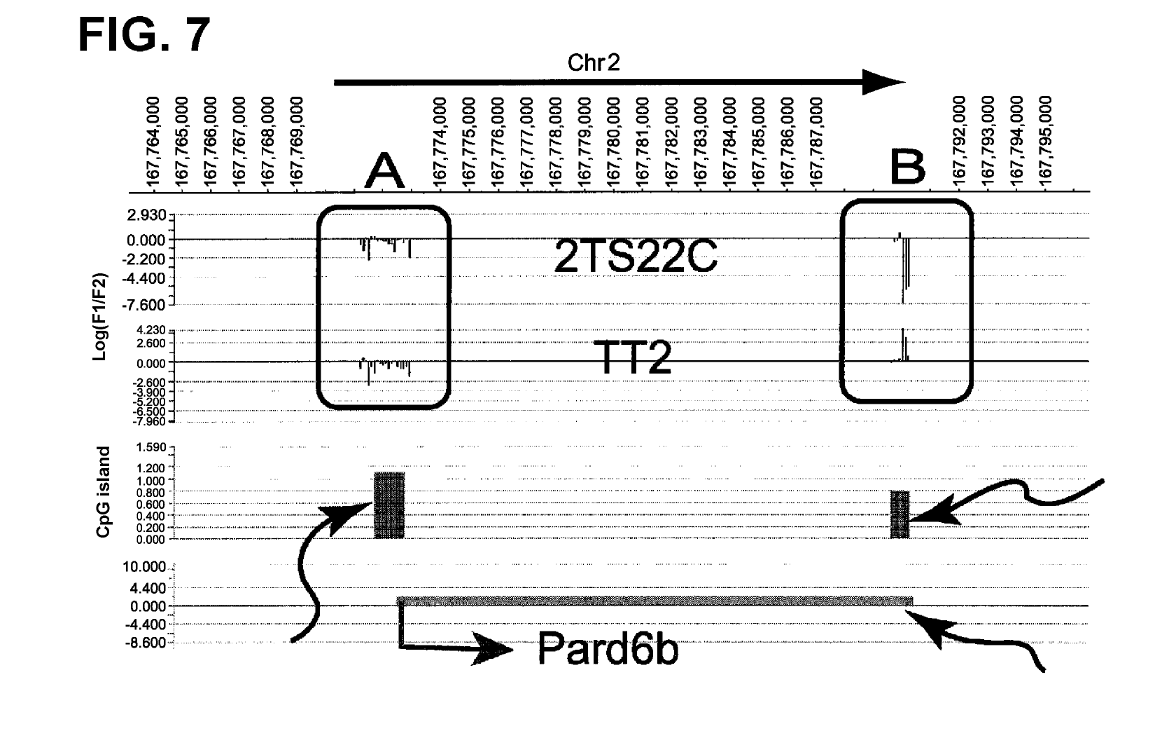

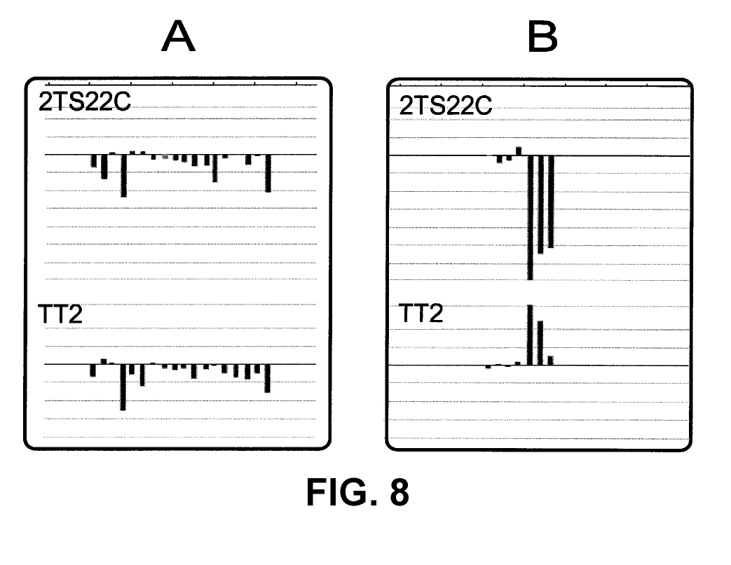

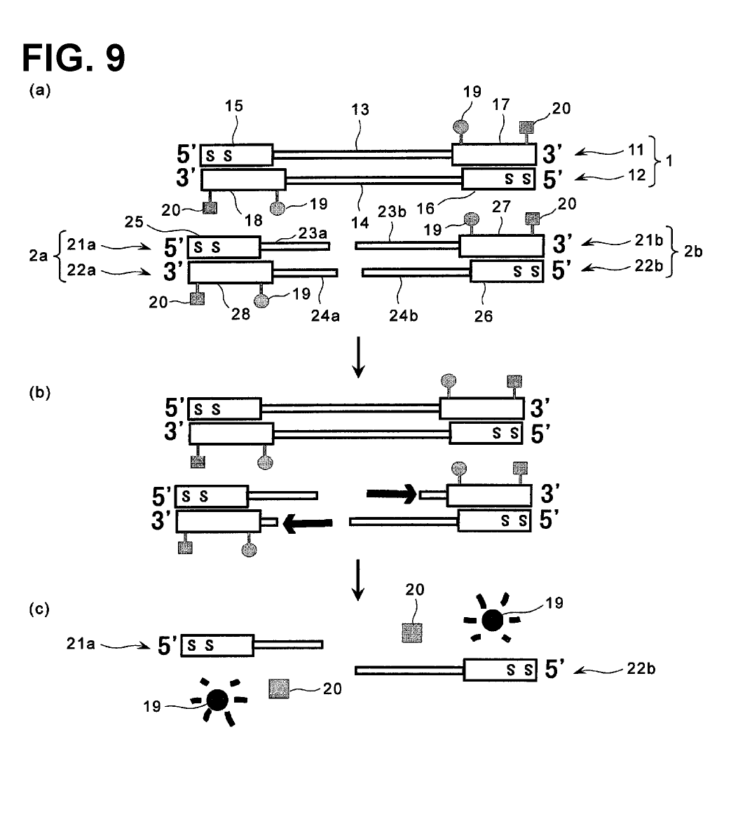

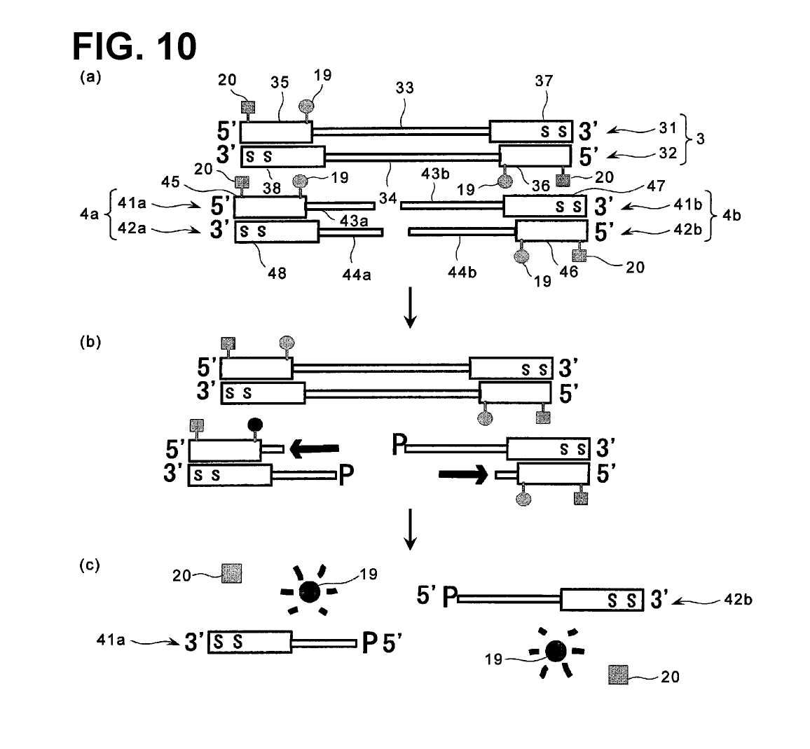

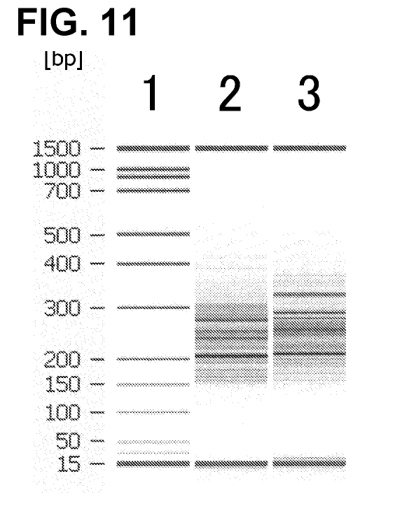

### BRIEF DESCRIPTION OF THE DRAWINGS

[FIG. 1] Explanatory diagram showing four DNA constructs (Ad+mC/mC+Ad, Ad+mC/C+Ad, Ad+C/mC+Ad, and Ad+C/C+Ad) obtained at step (2) on an avidin immobilizing carrier.

[FIG. 2] Explanatory diagram showing various operations (adaptor ligation, separation, amplification, etc.) at each DNA construct.

[FIG. 3] Drawing showing results of electrophoresis on PCR amplicon obtained in accordance with Embodiment 1 of the present invention in Working Example 1.

[FIG. 4] Graph showing results of carrying out DNA array analysis on fluorescently labeled PCR amplicon obtained at Working Example 1.

[FIG. 5] Explanatory diagram showing results obtained using a DNA array for analysis of mouse chromosome 11 gene Nmur2 (NM_{—}153079) to analyze PCR products respectively obtained by the McBT technique and the CRED technique.

[FIG. 6] Explanatory diagram showing results obtained using a DNA array for analysis of mouse chromosome 1 gene Fzd7 (NM_{—}008057) to analyze PCR products respectively obtained by the McBT technique and the CRED technique.

[FIG. 7] Explanatory diagram showing results obtained using a DNA array for analysis of mouse chromosome 2 gene Pard6b (NM_{—}021409) to analyze PCR products respectively obtained by the McBT technique and the CRED technique.

[FIG. 8] Explanatory diagram showing Areas A and B of FIG. 7 in enlarged fashion.

[FIG. 9] Explanatory diagram showing in schematic fashion a procedure for implementing a fluorophore and quencher technique employing 5′ to 3′ exonuclease.

[FIG. 10] Explanatory diagram showing in schematic fashion a procedure for implementing a fluorophore and quencher technique employing 3′ to 5′ exonuclease.

[FIG. 11] Drawing showing results of electrophoresis on PCR product obtained using the CRED technique.

### EMBODIMENTS FOR CARRYING OUT INVENTION

### 1. Methylation Analysis Methods in Accordance with the Present Invention

A method for analysis of methylation in accordance with the present invention is characterized in that it comprises at least:

(1) a step (hereinafter “MI restriction enzyme digest step”) in which analyte DNA is digested with methylation-insensitive restriction enzyme(s) (hereinafter “MI restriction enzyme”) that produce overhanging end(s) and that contain methylated cytosine or methylatable cytosine in recognition sequence(s) thereof;

(2) a step (hereinafter “adaptor ligation step”) in which adaptor(s) capable of regenerating recognition sequence(s) of the aforesaid methylation-insensitive restriction enzyme(s) is/are ligated to both ends of DNA fragment(s) obtained at step (1); and

(3) a step (hereinafter “MS restriction enzyme digest step”) in which DNA construct(s) obtained at step (2) is/are digested with methylation-sensitive restriction enzyme(s) (hereinafter “MS restriction enzyme”) that recognize the same recognition sequence(s) as the aforesaid methylation-insensitive restriction enzyme(s);

a plurality of embodiments being encompassed thereby depending on differences in handling of DNA construct(s) and/or digest product(s) after these have been obtained, the primary analytical results obtained, and so forth. Hereinafter, methylation analysis methods in accordance with the present invention that employ combination of MI restriction enzyme(s) and MS restriction enzyme(s) may be referred to as the McBT technique.

A method for analysis of methylation in accordance with Embodiment 1 of the present invention may comprise:

(1) a step in which analyte DNA is digested with MI restriction enzyme(s);

(2) a step in which adaptor(s) capable of regenerating MI restriction enzyme recognition sequence(s) is/are ligated to both ends of DNA fragment(s) obtained at step (1);

(3) a step in which DNA construct(s) obtained at step (2) is/are digested with MS restriction enzyme(s) that recognizes the same recognition sequence(s) as the aforesaid MI restriction enzyme(s);

(4) a step in which digest product(s) obtained at step (3) and amplification primer(s) capable of hybridization with adaptor(s) referred to in step (2) are used to amplify DNA; and

(5) a step in which DNA amplicon(s) obtained at step (4) is/are analyzed. Note that where the quantity of analyte DNA starting material is sufficient, it may be possible to eliminate the amplification step of step (4) and analyze digest product(s) obtained at step (3) without carrying out amplification.

The present embodiment makes it possible for only DNA fragments whose two ends are both methylated to be amplified, and for these to be identified.

Furthermore, a method for analysis of methylation in accordance with the present invention (hereinafter “Embodiment 1a”) may, where desired, in addition to the respective steps of the aforementioned Embodiment 1, further comprise:

(3a) a step in which DNA construct(s) obtained at step (2) is/are digested with methylation-dependent restriction enzyme(s) (e.g., McrBC; hereinafter “MD restriction enzyme”);

(4a) a step in which digest product(s) obtained at step (3a) and amplification primer(s) capable of hybridization with the adaptor(s) referred to in step (2) are used to amplify DNA; and

(5a) a step in which DNA amplicon(s) obtained at step (4a) is/are analyzed. Note that where the quantity of analyte DNA starting material is sufficient, it may be possible to eliminate the amplification step of step (4a) and analyze digest product(s) obtained at step (3) without carrying out amplification.

Note that as Embodiment 1a is an embodiment combining the McBT technique and the CRED technique, described below, detailed description will be given in the section on the CRED technique.

Furthermore, a method for analysis of methylation in accordance with the present invention may, as a negative control, further comprise, in addition to the respective steps of the aforementioned Embodiment 1 (or Embodiment 1a):

(3′) a step in which DNA construct(s) obtained at step (2) is/are digested with MI restriction enzyme(s) that recognize the same recognition sequence(s) as MI restriction enzyme(s) referred to in step (1);

(4′) a step in which digest product(s) obtained at step (3′) and amplification primer(s) referred to in step (4) are used to amplify DNA; and

(5′) a step in which DNA amplicon(s) obtained at step (4′) is/are analyzed. Note that where the quantity of analyte DNA starting material is sufficient, it may be possible to eliminate the amplification step of step (4′) and analyze digest product(s) obtained at step (3′) without carrying out amplification.

As the MI restriction enzyme used at step (3′), the same enzyme as the MI restriction enzyme used at step (1) may be employed, or a different enzyme may be employed, provided only that it is capable of recognizing the same recognition sequence. As will be described in detail below, by carrying out steps (3′) through (5′) it is possible to determine whether respective steps (1) through (3) are proceeding normally.

Furthermore, a method for analysis of methylation in accordance with the present invention may, for amplification of all DNA fragments as a comparative study, further comprise, in addition to the respective steps of the aforementioned Embodiment 1 (or Embodiment 1a; and preferably the respective steps of the aforementioned negative control):

(4″) a step in which DNA construct(s) obtained at step (2) and the amplification primer(s) referred to in step (4) are used to amplify DNA; and

(5″) a step in which DNA amplicon(s) obtained at step (4″) is/are analyzed. Note that where the quantity of analyte DNA starting material is sufficient, it may be possible to eliminate the amplification step of step (4″) and analyze DNA construct(s) obtained at step (2″) without carrying out amplification.

A method for analysis of methylation in accordance with Embodiment 2 of the present invention is primarily characterized by employment of an adaptor (hereinafter “first adaptor”) labeled with a substance capable of selective binding as the adaptor employed at step (2). Furthermore, the aforementioned Embodiment 2 may be further subdivided into two subembodiments (Embodiment 2a and Embodiment 2b) depending on whether or not the MS restriction enzyme digest treatment of step (3) is carried out while captured by a carrier.

Embodiment 2a, in which step (3) is not carried out while captured by a carrier, may comprise:

(1) a step in which analyte DNA is digested with MI restriction enzyme(s);

(2) a step in which first adaptor(s), which is/are labeled with substance(s) capable of selective binding and which is/are capable of regenerating MI restriction enzyme recognition sequence(s), is/are ligated to both ends of DNA fragment(s) obtained at step (1);

(3) a step in which DNA construct(s) obtained at step (2) is/are digested with MS restriction enzyme(s) that recognize the same recognition sequence(s) as the aforesaid MI restriction enzyme(s);

(4a) a step in which digest product(s) obtained at step (3) is/are separated based on presence or absence of the aforesaid label(s); and

(5a) a step in which one or both of the separated DNA fragment fractions is or are analyzed.

Furthermore, Embodiment 2b, in which step (3) is carried out while DNA construct(s) to be digested is/are captured by carrier(s), comprises:

(1) a step in which analyte DNA is digested with MI restriction enzyme(s);

(2) a step in which first adaptor(s), which is/are labeled with substance(s) capable of selective binding and which is/are capable of regenerating MI restriction enzyme recognition sequence(s), is/are ligated to both ends of DNA fragment(s) obtained at step (1);

(3) a step in which DNA construct(s) obtained at step (2) is or are, while captured by carrier(s) by way of the aforesaid labeled substance(s), digested with MS restriction enzyme(s) that recognize the same recognition sequence(s) as the aforesaid MI restriction enzyme(s);

(4b) a step in which the immobilizing carrier(s) used to carry out the enzyme digest treatment of step (3) is/are washed, and separation into a fraction containing DNA fragment(s) captured by the carrier(s) and a fraction containing DNA fragment(s) liberated from the carrier(s) is carried out; and

(5b) a step in which one or both of the separated DNA fragment fractions is or are analyzed.

The method for analysis of methylation in accordance with Embodiment 2 (including Embodiment 2a and Embodiment 2b) of the present invention makes it possible to carry out separation to obtain a fraction containing DNA fragments having at least one methylated end and a fraction containing DNA fragments whose two ends are both unmethylated, and for these to be amplified and identified. Furthermore, it is possible to further separate the aforesaid fraction containing DNA fragments having at least one methylated end into a fraction containing DNA fragments whose two ends are both methylated, and a fraction containing DNA fragments having only one methylated end, and to amplify and identify these, and it is moreover possible to determine which end is methylated.

Another method for analysis of methylation in accordance with the present invention is characterized in that it comprises at least:

(1) a step (hereinafter “fragmentation restriction enzyme digest step”) in which analyte DNA is digested with restriction enzyme(s);

(2) a step (hereinafter “adaptor ligation step”) in which adaptor(s) is/are ligated to both ends of DNA fragment(s) obtained at the aforesaid step (1); and

(3) a step (hereinafter “MD restriction enzyme digest step”) in which DNA construct(s) obtained at the aforesaid step (2) is/are digested with MD restriction enzyme(s) (e.g., McrBC);

a plurality of embodiments being encompassed thereby depending on whether or not there is combination with the aforesaid McBT technique, the primary analytical results obtained, and so forth. Hereinafter, methylation analysis methods in accordance with the present invention that use MD restriction enzyme(s) may be referred to as the CRED technique.

Furthermore, the aforesaid CRED technique may further comprise:

(4) a step in which digest product(s) obtained at step (3) and amplification primer(s) capable of hybridization with adaptor(s) referred to in step (2) are used to amplify DNA; and

(5) a step in which DNA amplicon(s) obtained at step (4) is/are analyzed. Note that where the quantity of analyte DNA starting material is sufficient, it may be possible to eliminate the amplification step of step (4) and analyze digest product(s) obtained at step (3) without carrying out amplification.

Below, description is given with respect to the McBT technique of the present invention, following which description is given with respect to the CRED technique of the present invention.

### McBT Technique

A method for analysis of methylation in accordance with the present invention (hereinafter meaning, in the present section, the McBT technique, unless otherwise stated) uses combination of methylation-sensitive restriction enzyme(s) (MS restriction enzyme(s)) and methylation-insensitive restriction enzyme(s) (MI restriction enzyme(s)) capable of recognizing identical recognition sequence(s). There is no particular limitation with respect to the aforesaid MI restriction enzyme, provided only that it produces an overhanging end and contains methylated cytosine or methylatable cytosine in the recognition sequence thereof; MspI, XmaI, and TaqI may be cited as examples. So long as it is capable of recognizing the same recognition sequence as the MI restriction enzyme, the cleavage site(s) and end configuration(s) produced by the aforesaid MS restriction enzyme may be the same as or different from those of the MI restriction enzyme; HpaII, NaeI, NgoMIV, or SmaI may be cited as examples for use with MspI; SmaI may be cited as an example for use with XmaI; and XhoI or ClaI may be cited as examples for use with TaqI. Recognition sequences and cleavage sites for these restriction enzymes are given in TABLE 1.

TABLE 1

| Restriction | Methylation | Recognition Sequence |
|---|---|---|
| Enzyme | Sensitivity | and Cleavage Site |
| MspI | MI | C:CGG |
| HpaII | MS | C:CGG |
| NaeI | MS | GCC:GGC |
| NgoMIV | MS | G:CCGGC |
| XmaI | MI | C:CCGGG |
| SmaI | MS | CCC:GGG |
| TaqI | MI | T:CGA |
| XhoI | MS | C:TCGAG |
| ClaI | MS | AT:CGAT |

Note that the MS restriction enzyme used in the present invention, so long as it is capable of recognizing the same recognition sequence as the MI restriction enzyme, need not perfectly match the recognition sequence, use being possible even where it merely contains the MI restriction enzyme recognition sequence. For example, when MspI (recognition sequence C:CGG) is used as MI restriction enzyme, NgoMIV (recognition sequence G:CCGGC), NaeI (recognition sequence GCC:GGC), or SmaI (recognition sequence CCC:GGG) may be used as MS restriction enzyme.

At step (1), i.e., the MI restriction enzyme digest step, of the methylation analysis method of the present invention, analyte DNA is digested with MI restriction enzyme. There is no particular limitation with respect to the DNA to which the methylation analysis method of the present invention may be applied, provided only that it is DNA capable of containing methylated cytosine or methylatable cytosine; artificially synthesized DNA, liberated DNA fragment mixtures present in biological samples or samples derived therefrom (e.g., blood, blood plasma, blood serum, urine, lymphatic fluid, spinal fluid, saliva, peritoneal fluid, amniotic fluid, mucus, milk, bile, gastric juice, artificial dialysis fluid obtained as result of dialysis treatment, etc.), and genomic DNA from cells (e.g., animal cells or plant cells) may be cited as examples. Where genomic DNA from cells is used, by carrying out treatment in advance with single-strand specific nuclease, e.g., mung bean nuclease, it is possible to eliminate loop regions or single-stranded regions produced during the course of artificial manipulation, or phenomena endogenous to the organism, permitting dramatic improvement in analytical accuracy, and permitting dramatic improvement in MI restriction enzyme digest efficiency.

Because the MI restriction enzyme used at step (1) is capable of cleaving all recognition sites regardless of whether or not the cytosine contained within the recognition sequence is methylated, carrying out the present step results in production of a mixture of four types of DNA fragments that differ based on whether or not there is methylation (i.e., whether or not methylated cytosine is present) at the two overhanging ends that are produced. The four types of DNA fragments are: DNA fragments in which both the upstream end and the downstream end are methylated (mC/mC); DNA fragments in which the upstream end is methylated but the downstream end is unmethylated (mC/C); DNA fragments in which the upstream end is unmethylated but the downstream end is methylated (C/mC); and DNA fragments in which both ends are unmethylated (C/C) [the symbol “mC” indicates that cytosine is methylated; the symbol “C” indicates that cytosine is unmethylated]. Note that what is referred to in the present specification as the upstream end and the downstream end of a DNA fragment, rather than being intended to be a particular direction, is a concept introduced for convenience of describing the present invention, and may be defined as appropriate by one practicing the present invention. For example, in the case of a structural gene, these might typically be defined as the ends respectively corresponding to the upstream direction and the downstream direction of the gene.

At step (2), i.e., the adaptor ligation step, of the methylation analysis method of the present invention, adaptor(s) is/are ligated to both ends of DNA fragment(s) obtained at step (1). In the present step, provided only that one end thereof is capable of bonding in complementary fashion to the overhanging end of the DNA fragment obtained at step (1), there is no particular limitation with respect to the adaptor employed, including the structure at the other end thereof. For example, when MspI (recognition sequence C:CGG) is used as MI restriction enzyme, and HpaII (recognition sequence C:CGG) is used as MS restriction enzyme as will be described below, because each of the two ends of the DNA fragment that is produced will have a 5′-CG overhanging structure as indicated below, the adaptor that is employed might be chosen such that it has a 5′-CG overhanging structure at one end. Furthermore, when the DNA fragment and the adaptor are ligated as a result of the ligation reaction as will be described below, the 5′ end of the DNA fragment sense strand could be made to form covalent bond(s) with the adaptor, and the 3′ end of the DNA fragment antisense strand could be prevented from forming covalent bond(s) with the adaptor. In such case, there is no objection to designing the adaptor so as to cause a gap to be produced between the adaptor and the 3′ end of the DNA fragment antisense strand.

| CHEMICAL FORMULA 1 | |
|---|---|
| 5′ 3′ | 5′ |
| (Adaptor) . . . C | p CGG . . . (DNA fragment sense strand) |
| (Adaptor) . . . GGC | C . . . (DNA fragment antisense strand) |
| 3′ 5′ | 3′ |

Note that where recognition sequences of the MI restriction enzyme and the MS restriction enzyme do not perfectly match, e.g., when MspI (recognition sequence C:CGG) is used as MI restriction enzyme and SmaI (recognition sequence CCC:GGG) is used as MS restriction enzyme, an adaptor might, for example, be designed as indicated below based upon consideration of the recognition sequence of the MS restriction enzyme.

| CHEMICAL FORMULA 2 | |
|---|---|
| 5′ 3′ | 5′ |
| (Adaptor) . . . CC | p CGGN . . . (DNA fragment sense strand) |
| (Adaptor) . . . GGGC | CN . . . (DNA fragment antisense strand) |
| 3′ 5′ | 3′ |

Below, notwithstanding that unless otherwise stated the methylation analysis method of the present invention will be primarily described in terms of examples in which the recognition sequences of the MI restriction enzyme and the MS restriction enzyme perfectly match, by making modifications as appropriate, one of skill in the art will be able to carry out the invention based on the descriptions contained in the present specification and common knowledge in the pertinent technical field even in situations in which the recognition sequences of the MI restriction enzyme and the MS restriction enzyme do not perfectly match.

Because the 5′ end of the DNA fragment obtained at step (1) is phosphorylated, the adaptor employed at step (2) may be such that the 5′ end of the oligonucleotide used for the antisense strand of the adaptor may be nonphosphorylated. At step (2), an excess amount of adaptor may be added to the DNA fragment mixture obtained at step (1), the ligation reaction being carried out at the point where complementary hydrogen bonds have formed between the DNA fragment mixture and the adaptor. Where the adaptor employed is such that the 5′ end of the oligonucleotide used for the antisense strand thereof is nonphosphorylated, because the 5′ end of the DNA fragment sense strand is phosphorylated, the aforesaid ligation reaction will cause ligation by way of covalent bonds between the 3′ end of the oligonucleotide used for the sense strand of the adaptor and the DNA fragment sense strand, but will not cause ligation by means of covalent bonds between the 5′ end of the oligonucleotide used for the antisense strand of the adaptor and the DNA fragment antisense strand 3′ end. On the other hand, where the adaptor employed is such that the 5′ end of the oligonucleotide used for the antisense strand thereof is phosphorylated, the aforesaid ligation reaction will cause both the sense strand and the antisense strand of the adaptor to each be ligated by way of covalent bonds to DNA fragments. Accordingly, where this is the case, the fill-in step becomes unnecessary and can be eliminated.

At step (2), the oligonucleotide used for the antisense strand of the adaptor may then be removed by heating until a temperature is reached that is the Tm of the adaptor or higher, or by lowering the Tm when in solution with DNA, and DNA polymerase may thereafter be used to extend the antisense strand of the DNA to obtain a DNA construct in which the adaptor sequence is ligated to both ends. The DNA fragment obtained at step (1) is obtained in the form of a mixture of four types that differ based on whether or not there is methylation at the two ends thereof, and the DNA construct obtained at step (2) is similarly obtained as a mixture of four types of DNA construct, i.e., a mixture of: DNA constructs in which both the upstream end and the downstream end are methylated (Ad+mC/mC+Ad); DNA constructs in which the upstream end is methylated but the downstream end is unmethylated (Ad+mC/C+Ad); DNA constructs in which the upstream end is unmethylated but the downstream end is methylated (Ad+C/mC+Ad); and DNA constructs in which both ends are unmethylated (Ad+C/C+Ad) [the symbol “Ad” means “adaptor”]. The MI restriction enzyme recognition sequence (e.g., C:CGG in the case of MspI) is regenerated at both ends of each of these DNA constructs.

Note that the restriction enzyme recognition sequence that is regenerated will, if the extension reaction is carried out in the presence of dNTPs, be such that cytosine in newly synthesized DNA strands is all unmethylated cytosine. Next, at step (3), i.e., the MS restriction enzyme digest step, if cytosine in only either the sense or antisense strand is methylated, then, in the event that an MS restriction enzyme that exhibits methylation sensitivity (i.e., cleavage is impeded by methylation) is used, methylation sensitivity will be such as to reflect the methylation state that existed in the original analyte DNA even when cytosine in newly synthesized DNA strands is unmethylated cytosine. On the other hand, if an MS restriction enzyme that exhibits methylation sensitivity is used when cytosine in both the sense and antisense strand is methylated, using 5-methyl-dCTP in place of dCTP when carrying out the extension reaction on the cytosine in the antisense strand of the adaptor will make it possible to cause methylation sensitivity to be such as to reflect the original methylation state of the analyte DNA.

As the aforesaid adaptor at step (2) of the methylation analysis method of the present invention, an adaptor that has been labeled (or modified) with substance(s) capable of selective binding (hereinafter “labeled adaptor”) may be used. As described in more detail below, use of such labeled adaptor(s) will make it possible to separate and purify DNA fragments based on whether or not there is methylation at the two overhanging ends that are produced.

At step (3), i.e., the MS restriction enzyme digest step, of the methylation analysis method of the present invention, DNA construct(s) obtained at step (2) is/are digested with MS restriction enzyme. The DNA construct mixture obtained at step (2) contains four types of DNA constructs that differ based on whether or not there is methylation at the two ends thereof, but MS restriction enzyme recognition sites are present at both ends of all of the DNA constructs.

Among the four types of DNA constructs, because the DNA constructs whose ends are both methylated (Ad+mC/mC+Ad) are not susceptible at all to cleavage by MS restriction enzyme, the digest product (Ad+mC/mC+Ad) is obtained. For DNA constructs having only one end that is methylated (Ad+mC/C+Ad and Ad+C/mC+Ad), the end that is methylated will not be susceptible to cleavage by MS restriction enzyme, but because the end that is unmethylated will be susceptible to cleavage by MS restriction enzyme, the adaptor sequence will be removed therefrom, and the digest products (Ad+mC/C and C/mC+Ad) will be obtained. Because DNA constructs whose ends are both unmethylated (Ad+C/C+Ad) will be susceptible to cleavage by MS restriction enzyme at both ends thereof, the adaptor sequence will be removed therefrom at both ends, and the digest product (C/C) will be obtained.

Next, at step (4), digest product(s) obtained at step (3) and amplification primer(s) capable of hybridization with adaptor(s) referred to in step (2) are used to amplify DNA. When one type of amplification primer capable of hybridization with adaptor(s) referred to in step (2) is used to carry out DNA amplification (e.g., PCR), only DNA fragments to which adaptors are ligated at both ends will be amplified, DNA fragments from which the adaptor sequence has been removed at one end, and DNA fragments from which the adaptor sequence has been removed at both ends, not being amplified. By analyzing the amplified DNA product, it is therefore possible at step (5) to identify DNA fragments whose two ends are both methylated.

Furthermore, in the present invention, as the aforesaid amplification primer, it is possible to use primer(s) comprising base sequence(s) capable of hybridization with the adaptor(s), and base sequence(s) capable of hybridization with gene sequence(s) flanking the aforesaid adaptor(s) (i.e., sequence(s) toward the end of the DNA fragment). Using such a primer will make it possible to target for amplification only a particular DNA fragment having the aforesaid gene sequence, and by analyzing the resulting amplicon it will be possible to identify whether or not the two ends of the particular DNA fragment are both methylated.

On the other hand, where an adaptor capable of selective binding is used and it is possible to separate and purify DNA groups depending on DNA methylation, because it is sufficient if it is possible to analyze for presence or absence of target DNA fragments in respective fractions purified from the fraction that is captured or not captured by the solid phase, primer(s) comprising base sequence(s) capable of hybridization with interior region(s) of analyte DNA fragment(s) may be used even when it/they do not contain portion(s) of adaptor sequence(s).

At step (3), note that where an MS restriction enzyme is employed for which the recognition sequence does not perfectly match [e.g., when MspI (recognition sequence C:CGG) is used as MI restriction enzyme, and SmaI (recognition sequence CCC:GGG) is used as MS restriction enzyme], because the end structure immediately prior to ligation of the adaptor might, for example, be as follows . . .

| CHEMICAL FORMULA 3 | |
|---|---|
| 5′ 3′ | 5′ |
| (Adaptor) . . . CC | p CGGN . . . (DNA fragment sense strand) |
| (Adaptor) . . . GGGC | CN . . . (DNA fragment antisense strand) |
| 3′ 5′ | 3′ |

. . . even where an end is unmethylated, it is only when the foregoing base N at the DNA fragment side is G (sense strand)/C (antisense strand) that it will be susceptible to cleavage by MS restriction enzyme; accordingly, when the foregoing base N is some other base, the adaptor will not be removed therefrom. In such case, by using an amplification primer capable of hybridization with the MS restriction enzyme recognition sequence (e.g., CCC:GGG if SmaI is used), it will be possible to amplify only the desired DNA fragments, i.e., DNA fragments whose two ends are both methylated.

At step (5), DNA amplicon(s) obtained at step (4), or digest product(s) obtained at step (3), is/are analyzed. As method(s) employed at step (5) for analysis of DNA amplicon or digest product, any of various known DNA analysis methods may be employed as appropriate; electrophoresis, DNA array analysis, use of a sequencer to analyze sequences, and so forth may be cited as examples. Furthermore, as method(s) for analysis of digest product(s) obtained at step (3), it is possible as described below to use fluorophore and quencher technique(s) not employing PCR amplification. Note that the term “analysis” as employed in analysis step(s) in accordance with the present invention, in addition to identification of DNA methylation state, base sequence, sequence direction, and so forth, also includes implementation of various operations that may be required therefor [e.g., DNA modification (e.g., adaptor attachment), separation, amplification, enzymatic treatment, and so forth].

As described above, method(s) in accordance with Embodiment 1 of the present invention including steps (1) through (5) make it possible for only DNA fragments whose two ends are both methylated to be amplified, and for these to be identified. Methylation analysis method(s) in accordance with the present invention may be such that, when practicing the aforesaid Embodiment 1, negative control(s) including steps (3′) through (5′), and/or amplification of all DNA fragments including steps (4″) through (5″), may be carried out.

At negative control step (3′), DNA construct(s) obtained at step (2) is/are digested with MI restriction enzyme(s) that recognize the same recognition sequence(s) as MI restriction enzyme(s) referred to in step (1). The MI restriction enzyme employed at step (3′) may be the same enzyme as the MI restriction enzyme employed at step (1), or may be a different enzyme that recognizes the same recognition sequence. The DNA construct obtained at step (2) includes four types of DNA construct, these being DNA constructs in which both the upstream end and the downstream end are methylated (Ad+mC/mC+Ad), DNA constructs in which the upstream end is methylated but the downstream end is unmethylated (Ad+mC/C+Ad), DNA constructs in which the upstream end is unmethylated but the downstream end is methylated (Ad+C/mC+Ad), and DNA constructs in which both ends are unmethylated (Ad+C/C+Ad); however, adaptor sequence(s) is/are present by way of MI restriction enzyme recognition site(s) at both ends of all of the DNA constructs. When these DNA constructs are digested with MI restriction enzyme, the adaptor sequences will be removed from both ends of all of the DNA constructs to produce the respective digest products (mC/mC, mC/C, C/mC, and C/C).

At negative control step (4′), digest product(s) obtained at step (3′) and amplification primer(s) referred to in step (4) are used to amplify DNA; and at step (5′), DNA amplicon(s) obtained at step (4′) is/are analyzed. Where respective steps (1) through (3) have proceeded normally, adaptor sequences will be removed from both ends of all digest product(s) obtained at negative control step (3′). Accordingly, even where an amplification primer capable of hybridization with the aforesaid adaptor is used to carry out DNA amplification (e.g., PCR), there will be no amplification of DNA whatsoever, and no DNA will be detected at step (5′).

On the other hand, in the event that respective steps (1) through (3) have not proceeded normally, e.g., where the restriction enzyme digest is incomplete, where a mismatch has occurred during the adaptor ligation reaction, or where, due to damage to the analyte DNA, the adaptor is ligated in an unintended configuration, because the MS restriction enzyme digest at step (3) will be incomplete, DNA amplification at step (4′) will result in production of unanticipated DNA amplicon(s), and DNA will be detected at step (5′).

Accordingly, by carrying out negative control(s) including steps (3′) through (5′) and confirming that DNA is not detected, it is possible to gain confidence in the fact that respective steps (1) through (3) of Embodiment 1 have proceeded normally and that the analytical results obtained are reliable.

At step (4″) for amplification of all DNA fragments as a comparative study, DNA construct(s) obtained at step (2) and amplification primer(s) referred to in step (4) are used to amplify DNA; and at step (5″), DNA amplicon(s) obtained at step (4″) is/are analyzed. The DNA construct obtained at step (2) includes four types of DNA construct, but adaptor sequence(s) is/are present by way of MI restriction enzyme recognition site(s) at both ends of all of the DNA constructs. Accordingly, when an amplification primer capable of hybridization with the aforesaid adaptor is used to carry out DNA amplification (e.g., PCR), all DNA constructs will be amplified. The DNA amplicon(s) obtained may be used as a comparative study for Embodiment 1.

As mentioned above, method(s) in accordance with Embodiment 1 of the present invention including steps (1) through (5) make it possible for only DNA fragments whose two ends are both methylated to be amplified, and for these to be identified. Methylation analysis methods in accordance with the present invention, through variations on Embodiment 1 as described above, make it possible to respectively separate, and furthermore to identify as such, each of the four types of DNA fragments that differ based on whether or not there is methylation at the two ends thereof, these being: DNA fragments in which both the upstream end and the downstream end are methylated (mC/mC); DNA fragments in which the upstream end is methylated but the downstream end is unmethylated (mC/C); DNA fragments in which the upstream end is unmethylated but the downstream end is methylated (C/mC); and DNA fragments in which both ends are unmethylated (C/C). Below, methods in accordance with the present invention will be further described in terms of an Embodiment 2a and an Embodiment 2b.

In the method for analysis of methylation in accordance with Embodiment 2a of the present invention, as the adaptor employed at step (2), besides use of labeled adaptor(s), it is possible by carrying out steps (1) through (3) of Embodiment 1, performing separation on the resulting digest product obtained based on whether or not the label is present [step (4a)], and analyzing the separated DNA fragments [step (5a)], to carry out separation to obtain a fraction containing DNA fragments having at least one methylated end and a fraction containing DNA fragments whose two ends are both unmethylated, and to identify these as such.

As the labeled adaptor, i.e., the adaptor labeled (or modified) with a substance capable of selective binding, examples which may be cited include an adaptor labeled with one of a pair of partner substances (e.g., combination of biotin and avidin, combination of an antigen and an antibody) capable of specific binding, or an adaptor having a reactive functional group (e.g., an NHS (N-hydroxysuccinimide) group that forms covalent bond(s) with an amino group, a maleimide group that forms covalent bond(s) with a thiol group, or a hydrazide group that forms covalent bond(s) with an aldehyde group) capable of selectively forming covalent bond(s). Furthermore, a substance serving as crosslinking agent for any of these and having within its molecular structure(s) capable of being cleaved by chemical treatment may be used.

Description will be given below in terms of an example in which biotin is used as the adaptor labeling substance. The digest product subjected to the separation operation at step (4a) contains four types of digest product (Ad+mC/mC+Ad, Ad+mC/C, C/mC+Ad, and C/C). At step (4a), after being made to contact an insoluble carrier (e.g., latex beads, magnetic particles, column carrier) in which, for example, avidin (capable of specific binding with biotin) has been immobilized, separation of the insoluble carrier from the reaction system makes it is possible to carry out separation to obtain a digest product fraction captured by the insoluble carrier (Ad+mC/mC+Ad, Ad+mC/C, and C/mC+Ad) and a digest product fraction not captured by the insoluble carrier (C/C).

At step (5a), ordinary method(s), e.g., by employing various DNA analysis methods mentioned above at step (5), or by carrying out various steps which will be described in more specific terms below, may be used to analyze the separated DNA fragments, i.e., DNA fragments whose two ends are both unmethylated and DNA fragments having at least one methylated end.

At step (5a) in the method for analysis of methylation in accordance with Embodiment 2a of the present invention, because DNA fragments whose two ends are both unmethylated (C/C), which are not captured by the insoluble carrier at step (4a), have an overhanging end produced by MI restriction enzyme treatment at step (3) at each of the two ends thereof, by ligating a suitable adaptor [e.g., the adaptor employed at step (2)] thereto, and thereafter employing an amplification primer capable of hybridization with the aforesaid adaptor, it is possible to amplify only the aforesaid DNA fragments.

Furthermore, at step (5a) in the method for analysis of methylation in accordance with Embodiment 2a of the present invention, from the fraction containing only DNA fragments having at least one methylated end, which is captured by the insoluble carrier at step (4a), depending on what is desired, it is possible to amplify only DNA fragments whose two ends are both methylated, or amplify only DNA fragments having only one methylated end.

For DNA fragments whose two ends are both methylated (Ad+mC/mC+Ad), because following MS restriction enzyme treatment at step (3) these will be such that adaptors are ligated at both ends, the aforesaid fraction may be used as template without the need for further alteration, and by using an amplification primer capable of hybridization with the aforesaid adaptor, it will be possible to amplify only the aforesaid DNA fragments.

On the other hand, for DNA fragments having only one end that is methylated (Ad+mC/C and C/mC+Ad), because following MS restriction enzyme treatment at step (3) these will be such that there is an overhanging end at one end, while an adaptor (first adaptor) remains at the other end, a suitable second adaptor might be ligated to the aforesaid overhanging end. Were the aforesaid fraction to be used as template, and an amplification primer capable of hybridization with the first adaptor and an amplification primer capable of hybridization with the second adaptor used in combination to carry out DNA amplification, because DNA fragments whose two ends are both methylated (i.e., DNA fragments in which the first adaptor is ligated at both ends) are also contained therein, this would result in amplification of the DNA fragments whose two ends are both methylated as well as the DNA fragments having only one methylated end. Accordingly, to amplify only the DNA fragments having only one methylated end, the aforesaid second adaptor might be labeled with a labeling substance (second labeling substance) different from the first adaptor labeling substance, and the second adaptor labeling substance utilized to remove DNA fragments whose two ends are both methylated, following which an amplification primer capable of hybridization with the first adaptor and an amplification primer capable of hybridization with the second adaptor would be used in combination to amplify only the DNA fragments having only one methylated end. Furthermore, fluorescently labeling one of the primers in advance will make it possible to identify which end is methylated (i.e., Ad+mC/C or C/mC+Ad), as will be described in detail below in connection with Embodiment 2b.

Note that the DNA fragments whose two ends are both methylated, which were removed as a result of utilization of the second adaptor labeling substance, are such that through use of an amplification primer capable of hybridization with the first adaptor it is possible to amplify only the aforesaid DNA fragments.

Except for the fact that adaptor(s) labeled with substance(s) capable of selective binding is/are employed as adaptor(s) at step (2) and the fact that MS enzyme digest treatment at step (3) is carried out while DNA construct(s) is/are captured by carrier(s), the methylation analysis method in accordance with Embodiment 2b of the present invention is carried out using the same operations as at steps (1) through (3) of Embodiment 1. By then washing the immobilizing carrier used to carry out MS enzyme digest treatment, separation can be carried out to obtain a fraction containing DNA fragments captured by the carrier and a fraction containing DNA fragments liberated from the carrier [step (4b)], and by analyzing the separated DNA fragments [step (5b)], separation can be carried out to obtain a fraction containing DNA fragments having at least one methylated end and a fraction containing DNA fragments whose two ends are both unmethylated, and to identify these as such. At the aforesaid step (5b), any of various steps described in connection with step (5a) may be carried out, or it is also possible to carry out an analysis method comprising:

(b1) a step in which second adaptor(s), which is/are labeled with substance(s) capable of selective binding and which is/are capable of regenerating MI restriction enzyme recognition sequence(s), is/are ligated to the free end side of the aforesaid DNA fragment(s) that remain captured by the immobilizing carrier(s) following washing thereof;

(b2) a step in which the DNA fragment(s) captured by the immobilizing carrier(s) is/are separated based on presence or absence of the aforesaid second label(s);

(b3) a step in which first amplification primer(s) capable of hybridization with the aforesaid first adaptor(s), and second amplification primer(s) capable of hybridization with the aforesaid second adaptor(s), are used to amplify DNA; and

(b4) a step in which DNA amplicon(s) obtained at the aforesaid step (b3) is/are analyzed.

The four types of DNA construct (Ad+mC/mC+Ad, Ad+mC/C+Ad, Ad+C/mC+Ad, and Ad+C/C+Ad) obtained at step (2) are shown at A) in FIG. 1 as they might appear when captured by an avidin immobilizing carrier; are shown at B) in FIG. 1 as they might appear in mid-reaction when, while still in that state, these are then subjected to digest treatment using MS restriction enzyme; and are shown at C) in FIG. 1 as they might appear following completion of the aforesaid enzyme digest treatment.

At FIG. 1, stars at respective DNA constructs 1 through 4 indicate where cytosine is methylated; arrows within DNA constructs indicate gene direction (i.e., arrows point from upstream to downstream); and black dots at either end of the DNA constructs indicate labeling substance (biotin). Note at FIG. 1 that avidin immobilized in carrier 5 has been omitted for convenience from the drawing.

DNA constructs 1, 2, 3, 4 in FIG. 1 are respectively the DNA construct (Ad+mC/mC+Ad), the DNA construct (Ad+mC/C+Ad), the DNA construct (Ad+C/mC+Ad), and the DNA construct (Ad+C/C+Ad).

While the description below in connection with Embodiment 2b is given in terms of an embodiment in which step(s) following MS restriction enzyme treatment at step (3) is/are carried out on a carrier, it is also possible in Embodiment 2b of the present invention to carry out step(s) following step (3) while dissociated from the carrier.

At step (3) in Embodiment 2b, when MS restriction enzyme treatment is performed while in the state shown at A) in FIG. 1, because cleavage does not occur at recognition site(s) where cytosine is methylated but does occur at recognition site(s) where cytosine is unmethylated, it will be the case, as shown at B) in FIG. 1, that digest product 1 (i.e., DNA construct 1; Ad+mC/mC+Ad) and digest product 2 (Ad+mC/C) will remain captured by the carrier but digest product 3 (C/mC+Ad) and digest product 4 (C/C) will be liberated from the carrier. However, because digest product 3 (C/mC+Ad), which has at this point been liberated, has a labeled adaptor at its downstream end, it can again be bound to avidin immobilizing carrier to ultimately result in a situation where, as shown at C) in FIG. 1, digest product 1 (i.e., DNA construct 1; Ad+mC/mC+Ad), digest product 2 (Ad+mC/C), and digest product 3 (C/mC+Ad) are captured by the carrier, but digest product 4 (C/C) is liberated from the carrier.

At step (4b) in Embodiment 2b, by washing the immobilizing carrier used to carry out the enzyme digest treatment of step (3), it is possible to separate the digest product (C/C) which is liberated, i.e., DNA fragments in which both ends are unmethylated, and DNA fragments captured by the immobilizing carrier, i.e., DNA fragments having at least one methylated end.

Then, at step (b1), the second adaptor(s), which is/are capable of regenerating MI restriction enzyme recognition sequence(s), is/are ligated to the free end side of the DNA fragment(s) that remain captured by the immobilizing carrier(s) following washing thereof. It is preferred that the aforesaid second adaptor be labeled using a partner substance (e.g., digoxigenin) different from the first adaptor labeling substance (first label).

As shown at C) in FIG. 1, because the first adaptor remains at the free end side of DNA fragment 1, both ends of which are methylated, the second adaptor is ligated only to DNA fragments 2 and 3, which each have only one methylated end. Ligation of adaptors to respective DNA fragments is as shown at FIG. 2. At FIG. 2, the first adaptor is shown in black, and the second adaptor is shown in gray.

At step (b2), which is next, the DNA fragment(s) captured by the immobilizing carrier by way of the first label is/are released from the carrier; and thereafter, DNA fragment 1 (Ad₁+mC/mC+Ad₁), DNA fragment 2 (Ad₁+mC/C+Ad₂), and DNA fragment 3 (Ad₂+C/mC+Ad₁), which exist in the form of a mixture, are separated based on presence or absence of the second adaptor labeling substance (second label). Taking as an example a situation in which digoxigenin is used as the second label, after causing this mixture to come in contact with an insoluble carrier in which anti-digoxigenin antibody has been immobilized, separation of the insoluble carrier from the reaction system makes it is possible to carry out separation to obtain a fraction containing DNA fragment 2 (Ad₁+mC/C+Ad₂) and DNA fragment 3 (Ad₂+C/mC+Ad₁), which are captured by the insoluble carrier, and a fraction containing DNA fragment 1 (Ad₁+mC/mC+Ad₁), which is not captured by the insoluble carrier.

Next, at step (b3), at least one of the first amplification primer, which is capable of hybridization with the aforesaid first adaptor, or the second amplification primer, which is capable of hybridization with the aforesaid second adaptor, is used to amplify DNA; and at step (b4), DNA amplicon(s) obtained at step (b3) is/are analyzed.

At step (b3), if the first primer and the second primer are used in combination to carry out DNA amplification (e.g., PCR), it will be possible to amplify only DNA fragments having only one methylated end (Ad₁+mC/C+Ad₂ and Ad₂+C/mC+Ad₁).

Furthermore, at step (b3), if the first primer and the second primer are used in combination, with one of the primers being fluorescently labeled in advance, this will make it possible to identify which end is methylated (i.e., Ad₁+mC/C+Ad₂ or Ad₂+C/mC+Ad₁).

For example, a procedure for carrying out determination in a situation in which the second primer is fluorescently labeled with Cy is shown schematically in FIG. 2. DNA fragment 2 in FIG. 2 is a DNA fragment in which the upstream end is methylated and the downstream end is unmethylated, and in which first adaptor 21 is ligated to the upstream end and second adaptor 22 is ligated to the downstream end, to form DNA fragment (Ad₁+mC/C+Ad₂). When the first primer 23, which hybridizes with the antisense strand of the first adaptor, and the second primer 24, which hybridizes with the antisense strand of the second adaptor and which is fluorescently labeled with Cy, are used in combination to carry out PCR, double-stranded DNA comprising fluorescently labeled antisense strand 26 and unlabeled sense strand 25 is amplified. When the amplified DNA is denatured, and this is applied to a DNA array in which DNA probe(s) that hybridize with the sense strand and DNA probe(s) that hybridize with the antisense strand are respectively arranged, a fluorescent signal is detected by probe(s) for the antisense strand.

On the other hand, DNA fragment 3 in FIG. 2 is a DNA fragment in which the upstream end is unmethylated and the downstream end is methylated, and in which second adaptor 32 is ligated to the upstream end and first adaptor 31 is ligated to the downstream end, to form DNA fragment (Ad₂+C/mC+Ad₁). When the first primer 33, which hybridizes with the antisense strand of the first adaptor, and the second primer 34, which hybridizes with the antisense strand of the second adaptor and which is fluorescently labeled with Cy, are used in combination to carry out PCR, double-stranded DNA comprising fluorescently labeled sense strand 35 and unlabeled antisense strand 36 is amplified. When the amplified DNA is denatured, and this is applied to a DNA array in which DNA probe(s) that hybridize with the sense strand and DNA probe(s) that hybridize with the antisense strand are respectively arranged, a fluorescent signal is detected by probe(s) for the sense strand.

Accordingly, when the second primer is fluorescently labeled with Cy, if a fluorescent signal is detected by probe(s) for the antisense strand then it will be possible to make the determination that this is a DNA fragment in which the upstream end is methylated and the downstream end is unmethylated, and if a fluorescent signal is detected by probe(s) for the sense strand then it will be possible to make the determination that this is a DNA fragment in which the upstream end is unmethylated and the downstream end is methylated.

The method for analysis of methylation in accordance with Embodiment 2b of the present invention not only makes it possible to separate DNA fragments having only one methylated end, but also, for example, makes it possible to respectively separate DNA fragments in which both ends are unmethylated at step (4b), and DNA fragments in which both ends are methylated at step (b2). Moreover, by selecting suitable primer(s), it will be possible to amplify these DNA fragment(s). For example, because DNA fragments whose two ends are both unmethylated have overhanging ends produced by MI restriction enzyme treatment at the two ends thereof, by ligating a suitable adaptor [e.g., the adaptor employed at step (2)] thereto, and thereafter employing an amplification primer capable of hybridization with the aforesaid adaptor, it is possible to amplify only the aforesaid DNA fragments. For DNA fragments whose two ends are methylated, because these will be such that the first adaptor is ligated to each of the two ends thereof, it will be possible by using an amplification primer capable of hybridization with the aforesaid adaptor to amplify only the aforesaid DNA fragments.

### CRED Technique

In a method for analysis of methylation in accordance with the present invention (hereinafter meaning, in the present section, the CRED technique, unless otherwise stated), at step (1), i.e., the fragmentation restriction enzyme digest step, there is no particular limitation with regard to the restriction enzyme(s) used to digest analyte DNA, it being possible to use any desired restriction enzyme(s) in accordance with the embodiment in question. Furthermore, there is no particular limitation with regard to the adaptor(s) used at step (2), i.e., the adaptor ligation step, it being possible to design any desired adaptor sequence(s) in accordance with the embodiment in question.

As analyte DNA used in the present step(s), the same as that which was used as mentioned above in connection with the McBT technique may be used, and/or it is also possible, where desired, to carry out treatment in advance with single-strand specific nuclease, e.g., mung bean nuclease.

Depending on the specific embodiment in question, the following may be cited as examples of a method for analysis of methylation in accordance with the present invention (CRED technique):

embodiment(s) carried out in combination with the McBT technique (i.e., the aforesaid Embodiment 1a);

embodiment(s) in which the CRED technique is carried out alone; and

embodiment(s) characterized by digestion with MD restriction enzyme(s) following circularization of analyte DNA that has been fragmented by restriction enzyme(s) (hereinafter “Embodiment 3”).

In an embodiment combining the McBT technique and the CRED technique, i.e., Embodiment 1a, the McBT technique might be carried out, while in parallel therewith, DNA construct(s) obtained as a result of carrying out MI restriction enzyme digest step (1) and adaptor ligation step (2) of the McBT technique is/are used to carry out:

(3a) a step in which DNA construct(s) obtained at step (2) is/are digested with MD restriction enzyme(s) (e.g., McrBC);

(4a) a step in which digest product(s) obtained at step (3a) and amplification primer(s) capable of hybridization with adaptor(s) referred to in step (2) are used to amplify DNA; and

(5a) a step in which DNA amplicon(s) obtained at step (4a) is/are analyzed. Note that where the quantity of analyte DNA starting material is sufficient, it may be possible to eliminate the amplification step of step (4a) and analyze digest product(s) obtained at step (3) without carrying out amplification.

As the MD restriction enzyme(s) employed in the methylation analysis method of the present invention, McrBC and/or McrA may, for example, be employed. In the case of linear DNA, McrBC recognizes a pair of PumC sequences [in the formula, Pu refers to purine base (A or G); mC refers to methylated cytosine] separated by an appropriate interval (approximately 40 to 3000 bases) [5′ . . . . PumC(N40-3000)PumC . . . 3′], and the DNA is cleaved at a single location near one of the PumC sites. Either single-stranded linear DNA or double-stranded linear DNA may serve as substrate; where double-stranded DNA is used, as presence of methylated cytosine in one of the strands will make it possible for cleavage to occur, hemimethylated locations can be cleaved.

On the other hand, in the case of circular DNA, regardless of whether it is single-stranded or double-stranded, if the PumC sequence exists at a single location, the DNA will be cleaved at a single location near that site.

The DNA construct(s) that is/are digested with MD restriction enzyme(s) at step (3a) of Embodiment 1a is/are DNA fragment group(s) obtained by digesting analyte DNA with MI restriction enzyme(s) and thereafter ligating adaptor(s) capable of regenerating the aforesaid MI restriction enzyme recognition sequence(s) thereto. When these DNA constructs are digested with McrBC, in the event that end region(s) (i.e., the MI restriction enzyme recognition sequence and the adaptor sequence) of the DNA construct do not contain the PumC sequence [e.g., when MspI (recognition sequence C:CGG) is used as MI restriction enzyme, and the base at the 5′ side upstream from that recognition sequence is neither A nor G], McrBC sensitivity/digest resistance will be determined based on the methylation state at only the interior regions of DNA fragments. That is, cleavage by McrBC will occur when there are two or more locations where PumC sequences exist in interior regions separated by appropriate interval(s). Since such cleaved fragment(s) will have at least one end thereof to which an adaptor is not ligated, they will not be amplified during the DNA amplification step (4a) which follows. On the other hand, when no PumC sequence exists in interior regions, or exists at only one location (or exists at two locations but these are separated by 3000 bases or more), cleavage by McrBC does not occur. Since such DNA fragment(s) will have adaptor(s) ligated to each of the two ends thereof, they will be amplified during the DNA amplification step (4a) which follows.

However, in the event that end region(s) of the DNA construct contain the PumC sequence [e.g., when MspI (recognition sequence C:CGG) is used as MI restriction enzyme, and the base at the 5′ side upstream from that recognition sequence is A or G], McrBC sensitivity/digest resistance will be determined based on overall methylation at interior regions as well as end regions of DNA fragments. That is, cleavage by McrBC will occur when there are two or more locations where PumC sequences exist in interior regions and/or end regions separated by appropriate interval(s). Since such cleaved fragment(s) will have at least one end thereof to which an adaptor is not ligated, they are not amplified during the DNA amplification step (4a) which follows. On the other hand, when no PumC sequence exists in interior regions and/or end regions, or exists at only one location (or exists at two locations but these are separated by 3000 bases or more), cleavage by McrBC does not occur. Since such DNA fragment(s) will have adaptor(s) ligated to each of the two ends thereof, they are amplified during the DNA amplification step (4a) which follows.

At the DNA amplification step (4a) which follows, the digest product(s) obtained and amplification primer(s) capable of hybridization with adaptor(s) referred to in the adaptor ligation step (2) of the McBT technique are used to amplify DNA. Regardless of whether or nor end regions contain PumC sequence(s), DNA fragments cleaved by McrBC at the MD restriction enzyme digest step (3a) will not be amplified, it being only DNA fragments which exhibit resistance to McrBC that are amplified.

At the DNA analysis step (5a), DNA amplicon(s) obtained at the aforesaid step (4a), or digest product(s) obtained at step (3), is/are analyzed. As method(s) employed at step (5a) for analysis of DNA amplicon or digest product, any of various known DNA analysis methods may be employed as appropriate; electrophoresis, DNA array analysis, use of a sequencer to analyze sequences, and so forth may be cited as examples. Furthermore, as method(s) for analysis of digest product(s) obtained at step (3), it is possible as described below to use fluorophore and quencher technique(s) not employing PCR amplification.

As mentioned above, the McBT technique makes it possible to evaluate methylation at both ends of DNA fragments based on MS restriction enzyme (e.g., HpaII) sensitivity/resistance. For example, when digest product produced by digestion with MS restriction enzyme (e.g., HpaII) is amplified with LM-PCR, only DNA fragments whose two ends are both methylated are amplified. When the resulting amplicon(s) is/are fluorescently labeled and applied to a DNA array, a fluorescent signal is detected for DNA fragments whose two ends are both methylated. Accordingly, the McBT technique makes it possible to carry out methylation determination with respect to only that cytosine which is located at end regions of DNA fragments.

On the other hand, when digest product produced by digestion with McrBC is amplified with LM-PCR, only DNA fragments which exhibit resistance to McrBC are amplified. Because resistance to McrBC depends on presence or absence of PumC sequence(s) only at interior regions (or at interior regions as well as end regions) of DNA fragments, it is possible to evaluate methylation at interior regions (or overall methylation at interior regions as well as end regions). For example, when digest product produced by digestion with McrBC is amplified with LM-PCR, and this is fluorescently labeled and thereafter applied to a DNA array, a fluorescent signal is detected for DNA fragments in which the McrBC recognition sequence does not exist at interior regions (or exists at neither interior regions nor end regions). Accordingly, the CRED technique makes it possible to carry out methylation determination with respect to that cytosine which is located at interior regions (or that which is located at interior regions and that which is located at end regions) of DNA fragments.

Note that with the CRED technique it is possible, through suitable choice of adaptor sequence(s) in accordance with the objectives of analysis, to select as appropriate whether only cytosine located at DNA fragment interior regions, exclusive of cytosine at end regions, will be targeted for analysis, or whether cytosine located at interior regions and end regions will simultaneously be targeted for analysis. That is, as mentioned above in connection with the aforesaid step (3a), if, for example, MspI (recognition sequence C:CGG) is used as MI restriction enzyme, and an adaptor sequence is designed that causes the base at the 5′ side upstream from that recognition sequence to be A or G, because the PumC sequence, which is the recognition sequence for McrBC, will be formed at end region(s), it will be possible to simultaneously target cytosine located at interior regions and end regions for analysis. However, even where MspI (recognition sequence C:CGG) has been used as MI restriction enzyme, if an adaptor sequence is designed that causes the base at the 5′ side upstream from that recognition sequence to be neither A nor G, it will be possible to target for analysis only cytosine located at DNA fragment interior regions, exclusive of cytosine at end regions.

Moreover, by combining information pertaining to fluorescent signal(s) (hereinafter “F1”) obtained using the McBT technique and information pertaining to fluorescent signal(s) (hereinafter “F2”) obtained using the CRED technique, complementary and redundant evaluation by the two enzymes will make it possible to more accurately carry out determination with respect to methylation of analyte DNA fragments. For example, the logarithm of the ratio between F1 and F2 [Log(F1/F2)] might be taken, a positive value for which meaning that the DNA fragment exhibits resistance to the MS restriction enzyme (e.g., HpaII) used in the McBT technique, and moreover, exhibits sensitivity to (digestibility by) the McrBC used in the CRED technique. That is, because this means that the two ends of the DNA fragment are both methylated, and in addition, that there is/are McrBC recognition sequence(s) at interior region(s), it will be possible to categorize that DNA fragment as a DNA fragment having a high level of methylation (hypermethylated DNA).

Conversely, if the value of Log(F1/F2) is negative, because this means that the DNA fragment exhibits sensitivity to the MS restriction enzyme and exhibits resistance to the McrBC, i.e., there is methylated cytosine in the restriction enzyme recognition sequence at only one of the two ends, or, there is no methylated cytosine at either end and there is no McrBC recognition sequence at interior regions, it will be possible to categorize that DNA fragment as a DNA fragment having a low level of methylation (hypomethylated DNA).

While the description above has been given in terms of Embodiment 1a (combining the McBT technique and the CRED technique), this being one embodiment of the methylation analysis method of the present invention, it is also possible to carry out the CRED technique alone.

Furthermore, whereas in Embodiment 1a digestion with MD restriction enzyme(s) was carried out while DNA was in linear form, in accordance with the methylation analysis method of the present invention it is also possible to carry out MD restriction enzyme digest while DNA is in circularized form (i.e., as at the aforesaid Embodiment 3).

A method for analysis of methylation in accordance with Embodiment 3 of the present invention may comprise:

(1) a step in which analyte DNA is digested with restriction enzyme(s);

(2) a step in which DNA fragment(s) obtained at the aforesaid step (1) is/are circularized;

(3) a step in which circular DNA obtained at the aforesaid step (2) is digested with MD restriction enzyme(s) (e.g., McrBC);

(4) a step in which digest product(s) obtained at step (3) is/are used to amplify circular DNA; and

(5) a step in which DNA amplicon(s) obtained at step (4) is/are analyzed. Note that where the quantity of analyte DNA starting material is sufficient, it may be possible to eliminate the amplification step of step (4) and analyze digest product(s) obtained at step (3) without carrying out amplification.

This Embodiment 3 exploits a property of McrBC with respect to circular DNA; i.e., regardless of whether it is single-stranded or double-stranded, if the PumC sequence exists at a single location, the DNA will be cleaved at a single location near that site, linearizing the DNA.

There being no particular limitation with regard thereto, the restriction enzyme(s) employed at step (1) of Embodiment 3 may be selected as appropriate in correspondence to the objectives of analysis. In Embodiment 3, the aforesaid restriction enzyme(s) is/are used to fragment analyte DNA at step (1), following which at step (2) this may be made to undergo self-circularization while still in two-stranded form, or this may be converted to single-stranded form by means of thermal denaturation or the like and thereafter made to undergo self-circularization through use of ssDNA ligase (e.g., CircLigase (TM) ssDNA ligase; manufactured by Epicentre). Recognition sequences cleaved by restriction enzyme(s) will be regenerated during circularization.

For example, when MspI (recognition sequence C:CGG) or TaqI (recognition sequence T:CGA) is used, even where that recognition sequence is regenerated in circular DNA it will not be recognized by McrBC. In such case, it will be possible to carry out analysis with respect to methylation only at interior regions of the DNA fragment(s) obtained by MspI digest. Accordingly, to analyze the DNA fragment(s) obtained through use of the aforesaid restriction enzyme with respect to methylation only at interior regions, exclusive of end regions, it is sufficient to use a restriction enzyme (e.g., MspI or TaqI) having a recognition sequence that will not serve as substrate for McrBC.

At step (3), circular DNA obtained at step (2) is digested with MD restriction enzyme; e.g., McrBC. If the PumC sequence exists at least one location in the circular DNA, that circular DNA will be cleaved, linearizing the DNA.

At step (4), which is next, the digest product obtained at step (3) is used to amplify only circular DNA through use of, e.g., rolling circle amplification utilizing phi29 DNA polymerase or other such amplification system capable of amplifying only circular DNA while still in circular DNA form. The circular DNA amplified at this step being DNA that is uncleaved by McrBC and that retains its circularization, only hypomethylated circular DNA is amplified.

At step (5), analysis of amplified circular DNA may employ as appropriate any of the various known DNA analysis methods which have been mentioned up to this point.

### Selective Removal of Enzyme-Digested Fragments

In method(s) for analysis of methylation in accordance with the present invention, whether in the context of the McBT technique or the CRED technique, restriction enzyme (MS restriction enzyme with the McBT technique and/or MD restriction enzyme with the CRED technique; hereinafter sometimes referred to as “DNA methylation analysis restriction enzyme(s)”) digest is used to produce DNA fragment(s) (having amplification adaptors at the two ends thereof) having resistance to the aforesaid enzyme(s) and cleavage product(s) (having at least one end at which there is no amplification adaptor) resulting from cleavage by the aforesaid enzyme(s), following which only DNA fragment(s) having amplification adaptors at the two ends thereof are amplified, e.g., by means of LM-PCR. Where, in accordance with the present invention, cleavage fragment(s) produced by DNA methylation analysis restriction enzyme digest is/are selectively removed before carrying out DNA amplification, this will make it possible to reduce nonspecific amplification reactions during amplification step(s), and make it possible to improve DNA amplification efficiency, or to maintain requisite DNA amplification efficiency while relaxing DNA amplification conditions.

As examples of methods for selectively removing cleavage fragment(s) produced by restriction enzyme digest, methods employing biotinylated adaptors, methods involving lysis and removal by nucleases, and so forth may be cited.

In a method employing biotinylated adaptor(s), biotinylated adaptor(s) might be used as adaptor(s) for ligation to DNA fragment(s), and after using MS restriction enzyme(s) or MD restriction enzyme(s) to carry out enzyme digest, the digest product(s) therefrom could be made to contact avidin-bearing carrier(s) (e.g., acquisition by means of avidin-coated PCR tube(s)), with DNA amplification being carried out while still in that state. This method would make it possible to easily remove cleavage fragment(s) not having biotinylated adaptors at both ends thereof from the reaction system.

In a method involving lysis and removal by nuclease(s), the fact that the 5′ end of DNA fragment(s) produced by restriction enzyme digest is phosphorylated is exploited. That is, as adaptor(s), adaptor(s) for which the 5′ end of the sense strand is nonphosphorylated, or adaptor(s) [phosphorothioate-type, ENA (Ethylene-bridged Nucleic Acids), LNA (Locked Nucleic Acid), etc.] for which the sense strand is designed to be exonuclease-resistant, or the like might be used, and through combination with any of various nucleases (e.g., λ exonuclease and/or Exonuclease I), only DNA fragment(s) targeted for removal would be lysed and removed.

For example, whereas λ exonuclease, which is a double-stranded DNA-digesting 5′ to 3′ exonuclease, efficiently lyses phosphorylated 5′ ends, it exhibits poor efficiency in digesting nonphosphorylated 5′ ends.

When the adaptor used is such that the 5′ end of the sense strand is nonphosphorylated, double-stranded DNA to which the adaptor has been ligated at both ends will not serve as substrate for λ exonuclease.

On the other hand, double-stranded DNA cleavage fragments produced by restriction enzyme digest, inasmuch as these are such that there is a phosphorylated 5′ end at both ends (for fragments that are derived from interior regions and that have been cleaved at two or more locations), or such that one end is phosphorylated (for DNA fragments that have been cleaved at one location), will serve as substrate for λ exonuclease.

Both the sense strand and the antisense strand of double-stranded DNA cleavage fragment having a phosphorylated 5′ end at both ends will be lysed and removed.

On the other hand, when the 5′ end at only one end is phosphorylated (i.e., when the 5′ end at an end produced by cleavage by enzyme digest is phosphorylated, and an adaptor is ligated to the end that is not the aforesaid end produced by cleavage), because the 5′ end of the adaptor sense strand is nonphosphorylated, that strand will not be lysed by λ exonuclease, but the opposing strand will be lysed by λ exonuclease. In such case, single-stranded DNA remains, which can be lysed and removed by single-stranded DNA-specific exonuclease(s) (e.g., Exonuclease I).

Note that even where the 5′ end is nonphosphorylated, while digest efficiency may be poor there is still lysis by λ exonuclease, so it is preferred for complete protection that adaptor(s) be employed that is/are designed to be exonuclease-resistant.

Where Escherichia coli Exonuclease III, which is a double-stranded DNA-specific 3′ to 5′ exonuclease, is employed in place of λ exonuclease, to protect double-stranded DNA to which adaptor(s) are ligated at both ends, adaptor(s) designed such that oligonucleotide(s) in antisense strand(s) are exonuclease-resistant might be used.

Double-stranded DNA having such adaptor(s) ligated at both ends thereof will not serve as substrate for Exonuclease III.

On the other hand, of the double-stranded DNA cleavage fragments produced by restriction enzyme digest, both the sense strand and the antisense strand of those cleavage fragments whose two ends are both cleaved ends produced by enzyme digest (i.e., in which adaptor(s) is/are not ligated at both ends) will be lysed and removed. Furthermore, for cleavage fragments in which one end is a cleaved end produced by enzyme digest and the aforesaid adaptor(s) is/are ligated at the other end, while the strand containing an antisense strand adaptor designed to be exonuclease-resistant will not be lysed by Exonuclease III, the opposing strand will be lysed by Exonuclease III. In such case, the single-stranded DNA which remains can be lysed and removed by single-stranded DNA-specific 5′ to 3′ exonuclease(s) (e.g., RecJ; manufactured by NEB).

Method for Analyzing Digest Product(s) from Digest Treatment with DNA Methylation Analysis Restriction Enzyme(s) without Carrying Out PCR Amplification (Fluorophore and Quencher Technique)

As has been described to this point, it would typically be the case that digest product(s) obtained as a result of treatment with DNA methylation analysis restriction enzyme(s) (i.e., MS restriction enzyme(s) in the context of the McBT technique and/or MD restriction enzyme(s) in the context of the CRED technique) might ordinarily be subjected to DNA amplification, following which any of various DNA analysis methods would be employed to carry out analysis of DNA amplicon(s). In method(s) for analysis of methylation in accordance with the present invention, by using fluorophore and quencher technique(s) as described below, it is also possible to directly analyze digest product(s) from digest treatment with DNA methylation analysis restriction enzyme(s) without carrying out PCR amplification.

In a fluorophore and quencher technique in accordance with the present invention, as described above in the section entitled “Selective Removal of Enzyme-Digested Fragments,” double-stranded DNA-digesting exonuclease might be used, and adaptor(s) appropriately designed in correspondence to the direction in which lysing proceeds might be used.

Either 5′ to 3′ exonuclease or 3′ to 5′ exonuclease(s) may be used as double-stranded DNA-digesting exonuclease, it being possible to use, e.g., λ exonuclease as 5′ to 3′exonuclease, and it being possible to use, e.g., Escherichia coli Exonuclease III as 3′ to 5′ exonuclease.

Below, an embodiment in which a 5′ to 3′ exonuclease is employed will be described with reference to FIG. 9, following which an embodiment in which a 3′ to 5′ exonuclease is employed will be described with reference to FIG. 10.

At FIG. 9, step (a) shows the situation that exists with digest products (DNA construct 1 and DNA construct lysates 2a and 2b) following digestion with DNA methylation analysis restriction enzyme(s); step (b) shows the situation that exists partway through digestion of the aforesaid digest products with 5′ to 3′ exonuclease; and step (c) shows the situation that exists in the reaction system following completion of digestion with 5′ to 3′ exonuclease. At step (c), note that DNA construct 1 continues to be present in the reaction system as shown elsewhere but has been omitted from the drawing for convenience.

Where 5′ to 3′ exonuclease is employed, oligonucleotides (e.g., phosphorothioate-type, ENA, and/or LNA) imparted with exonuclease resistance might be used as sense-side adaptors (adaptors 15, 16, 25, and 26 in FIG. 9); and oligonucleotides having fluorescent labels and quencher labels might be used as antisense-side adaptors (adaptors 17, 18, 27, and 28 in FIG. 9).

As shown at step (a) in FIG. 9, digest products resulting from digestion with DNA methylation analysis restriction enzyme(s) ordinarily include DNA construct 1 and DNA construct lysates 2a and 2b. DNA construct 1 is formed by ligation of adaptors (combination of sense-side adaptor 15 and antisense-side adaptor 18; combination of sense-side adaptor 16 and antisense-side adaptor 17) to both ends of DNA fragment(s) (sense strand 13 and antisense strand 14) obtained from digestion of analyte DNA (e.g., genomic DNA) with restriction enzyme(s). Note that regardless of whether in the context of the McBT technique or the CRED technique, when carrying out DNA amplification, there is no objection to presence of covalent bond(s) between adaptor antisense-side oligonucleotide(s) and DNA fragment(s) or to leaving this in the nicked state; however, in the fluorophore and quencher technique, if the exonuclease(s) used would recognize nick(s) and cause digestion, it will be necessary to carry out ligation using covalent bond(s). DNA construct 1 is present among the digest products as a moiety having resistance to DNA methylation analysis restriction enzyme(s), and lysates 2a and 2b are present thereamong as moieties having sensitivity to DNA methylation analysis restriction enzyme(s).

Note that prior to carrying out digestion with DNA methylation analysis restriction enzyme(s), to remove DNA fragment(s) to which adaptor(s) have not been ligated, it is preferred that these be lysed and removed by carrying out pretreatment with 5′ to 3′ exonuclease(s).

When 5′ to 3′ exonuclease is reacted with digest products that include DNA construct 1 and DNA construct lysates 2a and 2b, as shown at step (b), one strand 22a, 21b of each of the lysates 2a and 2b is lysed from the 5′ end thereof, while the remaining strand 21a, 22b thereof and DNA construct 1 do not undergo lysis. The strands 22a, 21b that undergo lysis by 5′ to 3′ exonuclease are such that, because adaptors 28, 27 ligated to the ends thereof are not exonuclease-resistant, following lysis of the DNA fragment portion 24a, 23b, the adaptors 28, 27 might also then be made to undergo lysis, liberating fluorescent substance 19 and quencher 20 into the reaction solution, as shown at step (c). By measuring the amount of fluorescent substance in this reaction solution, it is possible to evaluate sensitivity with respect to DNA methylation analysis restriction enzyme(s).

Where 3′ to 5′ exonuclease is employed, as shown in FIG. 10, oligonucleotides having quencher 20 and fluorescent labels 19 and might be used as sense-side adaptors 35, 36, 45, 46; and oligonucleotides imparted with exonuclease resistance might be used as antisense-side adaptors 37, 38, 47, 48.

In the present embodiment as well, prior to carrying out digestion with DNA methylation analysis restriction enzyme(s), to remove DNA fragment(s) to which adaptor(s) have not been ligated, it is preferred that these be lysed and removed by carrying out pretreatment with 3′ to 5′ exonuclease(s).

When, as shown at step (a) in FIG. 10, 3′ to 5′ exonuclease is reacted with digest products that include DNA construct 3 and DNA construct lysates 4a and 4b, as shown at step (b), one strand 41a, 42b of each of the lysates 4a and 4b is lysed from the 3′ end thereof, while the remaining strand 42a, 41b thereof and DNA construct 3 do not undergo lysis. The strands 41a, 42b that are lysed by 3′ to 5′ exonuclease are such that, because adaptors 45, 46 ligated to the ends thereof are not exonuclease-resistant, following lysis of the DNA fragment portion 43a, 44b, the adaptors 45, 46 might also then be made to undergo lysis, liberating fluorescent substance 19 and quencher 20 into the reaction solution, as shown at step (c). By measuring the amount of fluorescent substance in this reaction solution, it is possible to evaluate sensitivity with respect to DNA methylation analysis restriction enzyme(s).

At step (c), note that DNA construct 3 continues to be present in the reaction system as shown elsewhere but has been omitted from the drawing for convenience.

### 2. DNA Groups (Especially Circular DNA Groups) and Methods for the Manufacture Thereof as well as Methylation Analysis Methods (Methylation Profiling Analysis Methods) Utilizing Circular DNA Groups, in Accordance with the Present Invention

As has been described to this point, methylation analysis methods in accordance with the present invention make it possible, working from starting material comprising a mixture of DNA fragments obtained by digesting analyte DNA with MI restriction enzyme(s) that produce overhanging end(s) and that contain methylated cytosine or methylatable cytosine in recognition sequence(s) thereof, to separate and purify this into three groups of different types of DNA fragments that differ based on whether or not there is methylation (i.e., whether or not methylated cytosine is present) at the overhanging end that is produced at each of the two ends thereof, these being: a DNA fragment group in which both ends are methylated (i.e., a DNA fragment group in which methylated cytosine is present at both overhanging ends); a DNA fragment group in which only one end is methylated (a DNA fragment group in which methylated cytosine is present at only one of the overhanging ends); and a DNA fragment group in which both ends are unmethylated (a DNA fragment group in which methylated cytosine is present at neither of the overhanging ends).

Furthermore, with respect to the respective DNA fragments contained in the DNA fragment group in which only one end is methylated, because methylation analysis methods in accordance with the present invention make it possible to determine which end is methylated, it is possible to separate and purify the respective DNA fragments using known hybridization techniques utilizing DNA sequence(s) serving as probe(s) in DNA array(s) utilized in such determination. The respective separated and purified DNA fragments can be mixed to prepare formulations containing a DNA fragment group in which only the upstream end is methylated (a DNA fragment group in which methylated cytosine is present at only the upstream overhanging end) and/or a DNA fragment group in which only the downstream end is methylated (a DNA fragment group in which methylated cytosine is present at only the downstream overhanging end).

More specifically, in a method in which DNA fragments labeled with at least one of either a first adaptor or a second adaptor are subjected to denaturation treatment to obtain single-stranded DNA which is thereafter made available in the form of a DNA array or the like, arranging, in said DNA array, an oligo DNA probe for isolation of only one strand among base sequence(s) in respective strands (sense strand and antisense strand) constituting double-stranded DNA makes it possible to acquire only single-stranded DNA corresponding to base sequence(s) of the oligo DNA arranged in the DNA array [Direct Selection of Human Genomic Loci by Microarray Hybridization; Albert, T. J. et al., Nature Methods, 4 (11), pp. 903-905, 2007]. If single-stranded DNA obtained in this way is converted to the double-stranded form thereof in accordance with ordinary method(s), and the adaptor(s) is/are removed by digestion with MI restriction enzyme(s), because both ends of said double-stranded DNA will be overhanging ends, it will be possible to convert this to the circular form thereof.

Of the DNA groups in the present invention that are obtained using the McBT technique (which include a mixed DNA group containing circular DNA and linear DNA, and a circular DNA group made up of only circular DNA), the mixed DNA group containing circular DNA and linear DNA may be acquired by using DNA methylation analysis method(s) in accordance with the present invention to separate and purify these four DNA fragment groups, and respectively digesting these with MI restriction enzyme(s) that produce overhanging end(s) so as to cleave and eliminate all terminal adaptors, and thereafter performing ligation. Furthermore, following the aforesaid ligation, if exonuclease treatment is in addition carried out, it will be possible to acquire the circular DNA group made up of only circular DNA.

Alternatively, of the DNA groups in the present invention, DNA group(s) obtained by ligating one or more DNA fragment(s) in which methylated cytosine is present at neither of the two ends is/are such that, if steps (1) through (3) are carried out, and ligation is thereafter performed, because only DNA fragments in which both ends are overhanging ends will be efficiently ligated as compared with other DNA fragments, the desired DNA group(s) may be acquired.

The circular DNA group of the present invention, because it is circular, is capable of being amplified by strand displacement DNA polymerase (e.g., phi29 DNA polymerase) using random primer(s). Note that phi29 DNA polymerase is capable of amplifying both double-stranded circular DNA and single-stranded circular DNA, and the circular DNA group of the present invention may include both a double-stranded circular DNA group and a single-stranded circular DNA group. Here, by incorporating labeled nucleotide(s) (e.g., biotin-labeled dUTP) during amplification, it is also possible to obtain labeled amplicon(s) (DNA). In a situation where DNA strand displacement DNA polymerase is used and amplification is carried out using a randomly ligated circular DNA mixture as template, it will be possible to carry out amplification without bias with respect to gene length or the like, and with good fidelity with respect to relative ratios in which respective DNA fragments are present. Accordingly, with analyte DNA extracted from any of a wide variety of cells, e.g., various types of tissue, various types of cultured cells, and/or cells at various stages as starting material, manufacturing method(s) in accordance with the present invention might be employed to prepare circular DNA group(s) in accordance with the present invention, and amplification might be carried out by means of strand displacement DNA polymerase using random primer(s), following which analysis of the DNA amplicon(s) obtained would make it possible to carry out methylation profiling of analyte DNA. At such time, it is preferred that formulation(s) containing circular DNA group(s) in accordance with the present invention be prepared in advance from cells capable of being used as standard(s), because doing so will make it possible for these to be used in comparative studies against which analyte DNA may be judged during methylation analysis method(s) (especially analytic method(s) involving methylation profiling) in accordance with the present invention.

Of the DNA groups in the present invention that are obtained using the CRED technique (which include circular DNA group(s)), DNA group(s) made up of only DNA fragments that exhibit resistance with respect to methylation-dependent restriction enzyme(s) and that have restriction enzyme recognition sequences at both ends, wherein the aforesaid recognition sequences are all the same sequence, may be acquired using a manufacturing method in accordance with the present invention, comprising:

(1) a step in which analyte DNA is digested with restriction enzyme(s);

(2) a step in which adaptor(s) capable of regenerating recognition sequence(s) of the aforesaid restriction enzyme(s) is/are ligated to both ends of DNA fragment(s) obtained at the aforesaid step (1);

(3) a step in which DNA construct(s) obtained at the aforesaid step (2) is/are digested with methylation-dependent restriction enzyme(s); and

(4) a step in which amplification primer(s) capable of hybridization with adaptor(s) referred to in the aforesaid step (2) is/are used to amplify DNA, with digest product(s) obtained at the aforesaid step (3) serving as template(s).

Furthermore, circular DNA group(s) in accordance with the present invention characterized in that it/they are made up of only circular DNA not having methylation-dependent restriction enzyme recognition site(s) may be acquired using a manufacturing method in accordance with the present invention, comprising:

(1) a step in which analyte DNA is digested with restriction enzyme(s);

(2) a step in which DNA fragment(s) obtained at the aforesaid step (1) is/are circularized;

(3) a step in which circular DNA obtained at the aforesaid step (1) is digested with MD restriction enzyme(s); and

(4) a step in which only circular DNA is amplified, with digest product(s) obtained at the aforesaid step (3) serving as template(s).

Note that DNA fragment group(s) of the present invention is/are such that, where adaptors are present at both ends, the adaptors at both ends may be identical, or different adaptors may be used.

Furthermore, with respect to DNA group(s) of the present invention or respective DNA fragment(s) or DNA fragment group(s) identified in accordance with methylation analysis method(s) of the present invention, by arranging, on carrier(s) (e.g., plates, beads, hollow fibers, fibers, etc.), nucleic acid(s) respectively capable of hybridization with all or any portion of these DNA fragments, it is possible to obtain DNA array(s) in accordance with the present invention.

### WORKING EXAMPLES

Below, where the present invention is described more specifically in terms of working examples, these should not be understood as limiting the scope of the present invention.

### Working Example 1

Genomic DNA was prepared using a commercially available DNA purification kit (QIAamp DNA Micro Kit; manufactured by Qiagen), in accordance with the protocol provided therewith, from mouse ES cells (129/Ola-derived ES-E14TG2a; ATCC CRL-1821) and post-differentiation ES cells [cultured in medium without LIF on OP9 feeder cells; Sone et al., Arterioscler Thromb Vasc Biol. 2007 October; 27(10): 2127-34]. Taking 50 ng of this, treatment was carried out for 20 minutes at 37° C. using 50 units of mung bean nuclease. Following treatment, this solution was again purified using the aforesaid DNA purification kit. At this time, to completely remove alcohol content from column eluent/eluate, the pre-elution column was incubated for 5 minutes at 60° C., and this was eluted with 25 μL of 10 mmol/L Tris buffer solution (pH 8.0). Next, to 20 μL of this, 4 μL of 10× concentrated buffer solution for MspI digest use was added, distilled water being added to make this 40 μL. To this, methylation-insensitive (MI) restriction enzyme MspI [5 U if ordinary commercially available restriction enzyme; 1 μL of enzyme solution if FastCutter (manufactured by Fermentas)] was added, and digest was carried out for 2 hours at 37° C. The digest solution was then purified using a commercially available purification kit (ChargeSwitch PCR Clean-Up Kit; manufactured by Invitrogen). Elution was carried out using 10 μL of magnetic beads suspension to which 15 μL of 50 mmol/L Tris buffer solution (pH 8.0) was added.

4 μL of Adaptor 1 solution (LA1 solution) was added to the eluate, 19 μL of commercially available DNA ligation kit solution (DNA Ligation Kit MightyMix; manufactured by TaKaRa) was further added to this, and this was incubated for 30 minutes at 12° C. As a result, LA1, with formula as indicated below, . . .

CHEMICAL FORMULA 4
(Sequence No. 1)
5′-AGCACTCT CCAGCCTCTCACCGAC-3′
(Sequence No. 2)
3′-GGTCGGAGAGTGGCTGGC-5′

. . . is ligated to both ends of all DNA digest fragments obtained by MspI digestion of genomic DNA. Following termination of incubation for 30 minutes, with the reaction tube still at 12° C., Taq enzyme and enzyme reaction buffer solution (Expand High Fidelity PCR system; manufactured by Roche) were added, following which temperature of the reaction tube was increased to 72° C. to carry out the DNA fragment extension reaction. In more detail, following completion of the ligation reaction, a mixture of 11.4 μL of 10× buffer solution for PCR reaction use (containing Mg) and 102.6 μL of distilled water was added to the aforesaid ligation reaction solution, immediately following which incubation was carried out for 10 minutes at 72° C. As a result, strands of LA that are not covalently bonded are detached from DNA fragments by thermal melting, and the single-stranded portions formed are made into complete double-stranded structures during the extension reaction with the aforesaid Taq enzyme. During the extension reaction, even where cytosine within overhanging ends formed by aforesaid MspI digest is methylated, cytosine in the complementary strand newly synthesized during DNA extension will be unmethylated, such that methylated cytosine is preserved only at cytosine in one of the complementary strands of double-stranded DNA; i.e., the hemimethylated form is obtained. Next, the aforesaid reaction solution was again purified with a commercially available purification kit (ChargeSwitch PCR Clean-Up Kit; manufactured by Invitrogen) using 10 μL of magnetic beads suspension. DNA bound to magnetic beads was eluted with 30 μL of the eluent provided with the kit.

This was then divided into three aliquots, one of which was digested for 1 hour at 37° C. using 1 μL of MspI (manufactured by Fermentas) to make a reaction volume of 30 μL. Another aliquot was digested (decapped) for 1 hour at 37° C., under the same conditions as with MspI, using 1 μL of methylation-sensitive (MS) restriction enzyme HpaII (manufactured by Fermentas) to make a reaction volume of 30 μL. Next, the aforesaid respective MspI and HpaII enzyme digest solutions, and a total DNA fraction not subjected to enzyme digest, were respectively used as template DNA to carry out LM-PCR reactions under the following conditions.

### PCR Cycle Settings:

95° C., 2 minutes

+(95° C., 20 seconds; 71.4° C., 20 seconds; 73° C., 30 seconds)×18 cycles

+(95° C., 20 seconds; 70.0° C., 20 seconds; 73° C., 10 seconds)×26 cycles

+(95° C., 20 seconds; 70.0° C., 20 seconds; 73° C., 30 seconds)×8 cycles

+72° C., 5 minutes

52 cycles total, 0.5 μL template, 1 μL primer

| | |
|---|---|
| PCR primer: 5′-GCCTCTCACCGACCGG-3′ | (Sequence No. 3) |

PCR enzyme (kit): FastStart High Fidelity PCR System (manufactured by Roche)

Buffer used: Mg+ buffer provided with kit

TABLE 2
Composition of PCR Reaction Solution:

| | |
|---|---|
| 10x buffer solution (Mg+) | 2.0 |
| DMSO (final concentration 2%) | 0.4 |
| dNTP | 0.4 |
| PCR primer (RMspI-16PCR) | 1.0 |
| Sample | 0.5 |
| DNA polymerase (5 unit/μL) | 0.2 |
| Double-distilled water (DDW) | 15.5 |
| Total volume | 20.0 μL |

PCR products were analyzed using a bioanalyzer (BioAnalyzer; manufactured by Agilent) serving as analytic apparatus. Furthermore, analysis was carried out using commercially available analytic reagents (Agilent DNA 1000 Reagents; manufactured by Agilent). Results of electrophoresis of PCR products are shown at FIG. 3.

The respective lanes at FIG. 3 are as follows:

1=negative; 2=all DNA fragments; 3=ES cells; 4=differentiated cells (derived from ES cells).

Note that the negative (Lane 1) was produced by using MspI to remove LA and thereafter using the sample obtained as template to carry out PCR. For all DNA fragments (Lane 2), this was produced by using the sample as obtained following ligation of LA as template to carry out PCR. Furthermore, Lanes 3 and 4 were produced by redigesting the post-LA-ligation sample with HpaII, and using the sample obtained as template to carry out PCR.

### Working Example 2

Using established techniques, PCR products derived from mouse ES cells obtained at Working Example 1 were purified, and labeling with the fluorescent label Cy3 was carried out. Furthermore, PCR products derived from post-differentiation ES cells were purified in the same manner, and labeling with a different fluorescent label, Cy5, was carried out. A mouse genome DNA array (Mouse Promoter & CpG Island Tiling Array, cat# 00893; manufactured by NimbleGen) was used to carry out DNA array analysis of these labeled moieties.

Results of analysis for chromosome 1 are shown in FIG. 4. What is graphed is the ratio of post-differentiation ES cell fluorescence intensity over Cy3 fluorescence intensity (ES cells). ES cell gene fragments that are newly methylated post-differentiation produce increased Cy5 signal intensity at array probes corresponding to those gene fragments, manifesting as bars in the upper portion of the graph.

### Working Example 3

The procedure at Working Example 1 was followed to prepare respective solutions of DNA fragments having LA1 adaptors ligated thereto from two different mouse ES cell lines [2TS22C (RIKEN BioResource Center Cell Bank AES0125) and TT2 (same Bank AES0014)].

The several DNA fragment solutions were divided into four aliquots, one of which was digested with HpaII, an MS restriction enzyme (McBT technique); one of which was digested with McrBC (manufactured by NEB), an MD restriction enzyme (CRED technique); and one of which was digested with MspI, an MI restriction enzyme (negative control pursuant to McBT technique). The remaining aliquot was not subjected to enzyme treatment but was employed as positive control, representing total DNA, pursuant to the McBT technique. Digestion with McrBC was carried out by adding 20 units in a 40 μL reaction system, digestion being carried out for 3 to 5 hours at 37° C. Digestion with HpaII and MspI was carried out under the same conditions as described at Working Example 1.

Using the respective treatment solutions as template, the procedure described at Working Example 1 was followed to respectively carry out LM-PCR thereon. The PCR products obtained (including negative control and positive control) were subjected to electrophoresis to confirm that the reaction proceeded properly.

Respective PCR products obtained by carrying out LM-PCR with HpaII treatment solution or McrBC treatment solution serving as template were then labeled with green fluorescent dye (Alexa 555; manufactured by Invitrogen) or red fluorescent dye (Alexa 647; manufactured by Invitrogen) using established techniques. A mouse genome DNA array (Roche cat# 00893) was used to carry out DNA array analysis of these labeled moieties.

Analytic results for mouse chromosome 11 gene Nmur2 (NM_{—}153079) and regions upstream therefrom are shown in FIG. 5; analytic results for mouse chromosome 1 gene Fzd7 (NM_{—}008057) and regions upstream therefrom are shown in FIG. 6; and analytic results for mouse chromosome 2 gene Pard6b (NM_{—}021409) and regions upstream therefrom are shown in FIG. 7 through FIG. 8, (FIG. 8 being enlarged explanatory diagrams of Areas A and B shown in FIG. 7).

In the DNA array employed for analysis, probes generally 50mer in length were arranged at generally 50-base intervals; horizontal widths of and intervals between respective bars in Lane 2 in FIG. 5 and FIG. 6, horizontal widths of and intervals between respective dots in Lanes 3 and 4 in FIG. 5 and FIG. 6, and horizontal widths of and intervals between respective bars at log(F1/F2) insets in FIG. 7 and FIG. 8, respectively indicating probe length and probe interval.

In FIG. 5 and FIG. 6, the inverted triangles shown at Lane 1 indicate MspI recognition sites. Lane 3 and Lane 4 respectively indicate fluorescence intensities of green fluorescent dye (HpaII treatment; F1) and red fluorescent dye (McrBC treatment; F2), and Lane 2 is the logarithm of the ratio therebetween [Log(F1/F2)].

At FIG. 7 and FIG. 8, fluorescence intensities of green fluorescent dye (HpaII treatment; F1) and red fluorescent dye (McrBC treatment; F2) are omitted for convenience, only the logarithm of the ratio therebetween [Log(F1/F2)] being shown.

HpaII (MS restriction enzyme) methylation analysis is such that, because only DNA fragments whose two ends are both methylated are amplified, it is possible to carry out methylation determination with respect to only that cytosine which is located at end regions of DNA fragments. For example, in Lane 3 (green fluorescent dye) at FIG. 5 and FIG. 6, a strong fluorescent signal (F1) is detected for DNA fragments whose two ends are both methylated.

On the other hand, McrBC (MD restriction enzyme) methylation analysis permits evaluation of methylation with respect to cytosine located at DNA fragment interior regions, exclusive of cytosine at the aforesaid end regions. For example, in Lane 4 (red fluorescent dye) at FIG. 5 and FIG. 6, for DNA fragments in which the McrBC recognition sequence does not exist at interior regions (i.e., when there is no pair of PumC [in the formula, Pu refers to purine base (A or G); mC refers to methylated cytosine] sequences separated by an appropriate interval (40 to 3000 bases)), a strong fluorescent signal (F2) is detected.

Here, taking the logarithm of the ratio between F1 and F2 [Log(F1/F2)], if this value is positive (e.g., the region at the left side of the group of bars shown in Lane 2 at FIG. 5, or TT2 in Area B at FIG. 8), this means that the DNA fragment exhibits resistance to HpaII, and moreover, exhibits sensitivity to (digestibility by) McrBC, i.e., the two ends of the DNA fragment are both methylated, and in addition, there is/are McrBC recognition sequence(s) at interior region(s), so this DNA fragment may be categorized as a DNA fragment having a high level of methylation (hypermethylated DNA).

Conversely, if the value of Log(F1/F2) is negative (e.g., the region at the right side of the group of bars shown in Lane 2 at FIG. 6; or 2TS22C and TT2 in Area A, and 2TS22C in Area B, at FIG. 8), this means that the DNA fragment exhibits sensitivity to HpaII and exhibits resistance to McrBC, i.e., there is methylated cytosine in the restriction enzyme recognition sequence at only one of the two ends, or, there is no methylated cytosine at either end and there is no McrBC recognition sequence at interior regions, so this DNA fragment may be categorized as a DNA fragment having a low level of methylation (hypomethylated DNA).

Thus, by using the foregoing two enzymes to evaluate a particular DNA fragment in complementary and redundant fashion, it is possible to more accurately carry out determination with respect to methylation of analyte DNA fragments.

Whereas DNA methylation analysis employing restriction enzymes has heretofore been capable of carrying out determination with respect to methylation only at methylation-sensitive restriction enzyme recognition sites, because combination of the McBT technique and the CRED technique makes it possible, as has been described, for the same DNA fragment that served as analyte DNA for the McBT technique to be analyzed in similar fashion using the CRED technique, this method permits evaluation of methylation comprehensive of interior regions in analyte DNA, and overcomes deficiencies as have been present conventionally in restriction enzyme methods in which determination of methylation is carried out only with respect to restriction enzyme sites.

### Working Example 4

Using 4 mL of frozen pooled human blood sera (manufacturer=Nissui; product=L-Consera I-EX), genomic DNA within serum was purified with a commercially available purification kit (ChargeSwitch gDNA 1 mL Kit; manufactured by Invitrogen). This was digested for 1 hour with 5 units of MspI (manufactured by NEB) in accordance with ordinary method(s). This was purified using a purification kit [QIAquick Nucleotide Removal Kit (manufactured by Qiagen)], and an ultrafiltration membrane [Microcon Ultracel YM-30 (manufactured by Millipore)] was used to carry out equilibration (buffer exchange) with 25 mmol/L Tris-HCl (pH 8.0)/0.1 mmol/L EDTA on the DNA fragment solution obtained. The solution was concentrated to obtain 10 μL and said DNA solution was recovered, 4 μL of adaptor solution was added, 14 μL of ligase solution (MightyMix; manufactured by Takara Bio) was further added, and ligation was carried out for 1 hour at 12° C. The reaction solution was thereafter purified using a purification kit [QIAquick PCR Purification Kit (manufactured by Qiagen)], and an ultrafiltration membrane [Microcon Ultracel YM-30 (manufactured by Millipore)] was further used to equilibrate this with a buffer for restriction enzyme use [No. 2 (manufactured by NEB)] and concentrate this to obtain 20 μL. This was divided into two tubes containing 10 μL each; to only one of the tubes, 5 units of McrBC was added, and said solution was incubated for 4 hours at 37° C. Next, 3 μL of 10× buffer solution for exonuclease use [670 mmol/L Glycine-KOH (pH 9.4), 25 mmol/L MgCl₂, 10 mmol/L DTT, 0.5 mg/mL BSA] was added, distilled water was further added to obtain a total volume of 30 μL, and in addition, λ exonuclease (5 units) and Exonuclease I (20 units) were added, and this was incubated for 1 hour at 37° C., and was further incubated for 20 minutes at 75° C. Said solution was then made to serve as template, and the adaptor and oligo DNA primer employed at Working Example 1 were used to carry out LM-PCR. As a result, sufficient DNA amplicons were confirmed (FIG. 11) by electrophoresis (capillary electrophoresis using BioAnalyzer). At FIG. 11, Lane 1 is molecular weight markers, Lane 2 is PCR amplicons when total DNA (i.e., no McrBC digest) was used as template, and Lane 3 is PCR amplicons when McrBC-treated DNA was used as template. Quantities of these were sufficient to also permit DNA array analysis.

TABLE 3
PCR Conditions

| | | |
|---|---|---|
| 10x buffer solution | 2.0 | μL |
| DMSO (final concentration 2%) | 0.4 | μL |
| dNTP | 0.4 | μL |
| Primer (RMspI-16PCR) | 1.0 | μL (50 pmole/μL) |
| Template DNA solution | 1.0 | μL |
| DNA polymerase (5 U/μL) | 0.3 | μL (FastStart; mfd. by Roche) |
| Double-distilled water (DDW) | 14.9 | μL |
| Total volume | 20.0 | μL |

75° C., 10 minutes+95° C., 2 minutes

+(95° C., 20 seconds; 71.8° C., 15 seconds; 73° C., 30 seconds)×18 cycles

+(95° C., 20 seconds; 70.0° C., 20 seconds; 73° C., 10 seconds)×26 cycles

+(95° C., 20 seconds; 69.8° C., 20 seconds; 73° C., 30 seconds)×10 cycles

+72° C., 5 minutes

### INDUSTRIAL UTILITY

The present invention may be applied to DNA methylation analysis applications.

While the present invention has been described above in terms of particular embodiments, variations and improvements that would be obvious to one of skill in the art are included within the scope of the present invention.

### SEQUENCE LISTING FREE TEXT

Respective base sequences listed in the Sequence Listing as Sequence Nos. 1 and 2 are Adaptor 1. The base sequence listed in the Sequence Listing as Sequence No. 3 is PCR primer.

What is claimed is:

1. A method for analyzing methylation of DNA analyte, the method characterized in that it comprises:
(1) a first MI restriction enzyme digest step in which analyte DNA is digested with one or more first methylation-insensitive restriction enzymes that produce one or more overhanging ends and that contain methylated cytosine and/or methylatable cytosine in one or more recognition sequences thereof;
(2) a first adaptor ligation step in which one or more first adaptors capable of regenerating at least one of the recognition sequence or sequences of the first methylation-insensitive restriction enzyme or enzymes is or are ligated to both ends of one or more DNA fragments obtained at the first MI restriction enzyme digest step; and
(3) an MS restriction enzyme digest step in which one or more DNA constructs obtained at the first adaptor ligation step is or are digested with one or more methylation-sensitive restriction enzymes that recognize the same recognition sequence or sequences as at least a portion of the first methylation-insensitive restriction enzyme or enzymes.

2. A DNA methylation analysis method according to claim 1 further comprising:
an analysis step in which one or more first digest products obtained at the MS restriction enzyme digest step, and/or one or more first DNA amplicons obtained by amplification using one or more amplification primers capable of hybridization with at least one of the first adaptor or adaptors when at least one of the first digest product or products serves as template or templates, is or are analyzed.

3. A DNA methylation analysis method according to claim 2 further comprising:
a second MI restriction enzyme digest step in which at least one of the DNA construct or constructs obtained at the first adaptor ligation step is digested with one or more second methylation-insensitive restriction enzymes that recognize the same recognition sequence or sequences as at least a portion of the first methylation-insensitive restriction enzyme or enzymes; and
an analysis step in which one or more second digest products obtained at the second MI restriction enzyme digest step, and/or one or more second DNA amplicons obtained by amplification using at least one of the amplification primer or primers when at least one of the second digest product or products serves as template, is analyzed.

4. A DNA methylation analysis method according to claim 2 further comprising:
an analysis step in which at least one of the DNA construct or constructs obtained at the first adaptor ligation step, and/or or one or more third DNA amplicons obtained by amplification using at least one of the amplification primer or primers when at least one of the DNA construct or constructs serves as template, is analyzed.

5. A DNA methylation analysis method according to claim 1 wherein at least one of the first adaptor or adaptors is labeled with one or more first substances capable of selective binding.

6. A DNA methylation analysis method according to claim 5 further comprising:
(4a) a separation step in which one or more first digest products obtained at the MS restriction enzyme digest step is or are separated into two DNA fragment fractions based on presence or absence of at least one of the first labeling substance or substances; and
(5a) a step in which one or both of the separated DNA fragment fractions is or are analyzed.

7. A DNA methylation analysis method according to claim 5
wherein the MS restriction enzyme digest step is carried out while at least one of the DNA construct or constructs is captured by one or more immobilizing carriers by way of at least one of the first labeling substance or substances; and
wherein the DNA methylation analysis method further comprises:
(4b) a separation step in which at least one of the carrier or carriers is washed to carry out separation into a DNA fragment fraction captured by at least one of the carrier or carriers, and a DNA fragment fraction liberated from at least one of the carrier or carriers; and
(5b) a step in which one or both of the separated DNA fragment fractions is or are analyzed.

8. A DNA methylation analysis method according to claim 1 further comprising:
an MD restriction enzyme digest step in which at least one of the DNA construct or constructs obtained at the first adaptor ligation step is digested with one or more methylation-dependent restriction enzymes; and
an analysis step in which one or more third digest products obtained at the MD restriction enzyme digest step, and/or one or more fourth DNA amplicons obtained by amplification using one or more amplification primers capable of hybridization with at least one of the first adaptor or adaptors when at least one of the third digest product or products serves as template, is analyzed.

9. A DNA methylation analysis method according to claim 8 wherein at least one result of the analysis step is used to determine whether at least one DNA fragment obtained at the first MI restriction enzyme digest is:
(a) DNA exhibiting sensitivity with respect to a methylation-dependent restriction enzyme, and having methylated cytosine present in a restriction enzyme recognition sequence at each of the two ends thereof;
(b) DNA exhibiting digest resistance with respect to a methylation-dependent restriction enzyme, and having methylated cytosine present in a restriction enzyme recognition sequence at each of the two ends thereof;
(c) DNA exhibiting sensitivity with respect to a methylation-dependent restriction enzyme, and having methylated cytosine present in a restriction enzyme recognition sequence at only one of the two ends thereof or having methylated cytosine present at neither of the two ends thereof; and/or
(d) DNA exhibiting digest resistance with respect to a methylation-dependent restriction enzyme, and having methylated cytosine present in a restriction enzyme recognition sequence at only one of the two ends thereof or having methylated cytosine present at neither of the two ends thereof.

10. A DNA methylation analysis method according to claim 1 wherein the analyte DNA is treated with single-strand specific nuclease before the first MI restriction enzyme digest step is carried out.

11. A method of manufacturing:
(I′) a DNA group obtained by ligation of one or more DNA fragments, each of which DNA fragment has at each of the two ends thereof a recognition sequence containing methylated cytosine or methylatable cytosine, methylated cytosine being present at each of the two recognition sequences;
(II′) a DNA group obtained by ligation of one or more DNA fragments, each of which DNA fragment has at each of the two ends thereof a recognition sequence containing methylated cytosine or methylatable cytosine, methylated cytosine being present at only one of the two recognition sequences; and/or
(III′) a DNA group obtained by ligation of one or more DNA fragments, each of which DNA fragment has at each of the two ends thereof a recognition sequence containing methylated cytosine or methylatable cytosine, methylated cytosine being present at neither of the two recognition sequences;
the method comprising:
(A) a step in which a DNA methylation analysis method according to claim 1 is used to acquire:
(i) a DNA fragment group containing only DNA fragments, each of which DNA fragment has at each of the two ends thereof a recognition sequence containing methylated cytosine or methylatable cytosine, methylated cytosine being present at each of the two recognition sequences;
(ii) a DNA fragment group containing only DNA fragments, each of which DNA fragment has at each of the two ends thereof a recognition sequence containing methylated cytosine or methylatable cytosine, methylated cytosine being present at only one of the two recognition sequences; and/or
(iii) a DNA fragment group containing only DNA fragments, each of which DNA fragment has at each of the two ends thereof a recognition sequence containing methylated cytosine or methylatable cytosine, methylated cytosine being present at neither of the two recognition sequences; and
(B) a step in which the acquired DNA fragment group or groups is or are digested with at least one of the first methylation-insensitive restriction enzyme or enzymes to cleave and eliminate all terminal adaptors before carrying out the aforesaid DNA fragment ligation or ligations.

12. A DNA group manufacturing method according to claim 11 wherein the DNA group or groups is or are circular DNA groups containing only circular DNA; and
wherein exonuclease treatment is further carried out following the aforesaid DNA fragment ligation or ligations.

13. A method of manufacturing:
(I′) a DNA group obtained by ligation of one or more DNA fragments, each of which DNA fragment has at each of the two ends thereof a recognition sequence containing methylated cytosine or methylatable cytosine, methylated cytosine being present at each of the two recognition sequences;
(IIa′) a DNA group obtained by ligation of one or more DNA fragments, each of which DNA fragment has at each of the two ends thereof a recognition sequence containing methylated cytosine or methylatable cytosine, methylated cytosine being present at only the upstream recognition sequence;
(IIb′) a DNA group obtained by ligation of one or more DNA fragments, each of which DNA fragment has at each of the two ends thereof a recognition sequence containing methylated cytosine or methylatable cytosine, methylated cytosine being present at only the downstream recognition sequence; and/or
(III′) a DNA group obtained by ligation of one or more DNA fragments, each of which DNA fragment has at each of the two ends thereof a recognition sequence containing methylated cytosine or methylatable cytosine, methylated cytosine being present at neither of the two recognition sequences;
the method characterized in that it comprises:
(A) a step in which a DNA methylation analysis method according to claim 1 is used to acquire:
(i) a DNA fragment group containing only DNA fragments, each of which DNA fragment has at each of the two ends thereof a recognition sequence containing methylated cytosine or methylatable cytosine, methylated cytosine being present at each of the two recognition sequences;
(iia) a DNA fragment group containing only DNA fragments, each of which DNA fragment has at each of the two ends thereof a recognition sequence containing methylated cytosine or methylatable cytosine, methylated cytosine being present at only the upstream recognition sequence;
(iib) a DNA fragment group containing only DNA fragments, each of which DNA fragment has at each of the two ends thereof a recognition sequence containing methylated cytosine or methylatable cytosine, methylated cytosine being present at only the downstream recognition sequence; and/or
(iii) a DNA fragment group containing only DNA fragments, each of which DNA fragment has at each of the two ends thereof a recognition sequence containing methylated cytosine or methylatable cytosine, methylated cytosine being present at neither of the two recognition sequences; and
(B) a step in which the acquired DNA fragment group or groups is or are digested with at least one of the first methylation-insensitive restriction enzyme or enzymes to cleave and eliminate all terminal adaptors before carrying out the aforesaid DNA fragment ligation or ligations.

14. A DNA group manufacturing method according to claim 13 wherein the DNA group or groups is or are circular DNA groups containing only circular DNA; and
wherein exonuclease treatment is further carried out following the aforesaid DNA fragment ligation or ligations.

15. A DNA methylation analysis method according to claim 7 further comprising:
an MD restriction enzyme digest step in which at least one of the DNA construct or constructs obtained at the first adaptor ligation step is digested with one or more methylation-dependent restriction enzymes; and
an analysis step in which one or more third digest products obtained at the MD restriction enzyme digest step, and/or one or more fourth DNA amplicons obtained by amplification using one or more amplification primers capable of hybridization with at least one of the first adaptor or adaptors when at least one of the third digest product or products serves as template, is analyzed.

16. A DNA methylation analysis method according to claim 6 wherein the analyte DNA is treated with single-strand specific nuclease before the first MI restriction enzyme digest step is carried out.

17. A DNA methylation analysis method according to claim 7 wherein the analyte DNA is treated with single-strand specific nuclease before the first MI restriction enzyme digest step is carried out.

18. A DNA methylation analysis method according to claim 8 wherein the analyte DNA is treated with single-strand specific nuclease before the first MI restriction enzyme digest step is carried out.

19. A DNA methylation analysis method according to claim 3 further comprising:
an analysis step in which at least one of the DNA construct or constructs obtained at the first adaptor ligation step, and/or or one or more third DNA amplicons obtained by amplification using at least one of the amplification primer or primers when at least one of the DNA construct or constructs serves as template, is analyzed.

20. A DNA methylation analysis method according to claim 2 further comprising:
an MD restriction enzyme digest step in which at least one of the DNA construct or constructs obtained at the first adaptor ligation step is digested with one or more methylation-dependent restriction enzymes; and
an analysis step in which one or more third digest products obtained at the MD restriction enzyme digest step, and/or one or more fourth DNA amplicons obtained by amplification using at least one of the amplification primer or primers when at least one of the third digest product or products serves as template, is analyzed.


SEQUENCE LISTING (3 sequences)

SEQ ID NO: 1 - DNA, 24 bp, artificial sequence
  Description of Artificial Sequence adaptor 1
agcactctccagcctctcaccgac

SEQ ID NO: 2 - DNA, 18 bp, artificial sequence
  Description of Artificial Sequence adaptor 1
cggtcggtgagaggctgg

SEQ ID NO: 3 - DNA, 16 bp, artificial sequence
  Description of Artificial Sequence PCR primer
gcctctcaccgaccgg